(19) United States
(12) Patent Application Publication
(10) Pub. No.: US 2010/0143960 A1
(43) Pub. Date: Jun. 10, 2010

(54) CYANINE DERIVATIVES, FLUORESCENT CONJUGATES CONTAINING SAME AND USE THEREOF

(75) Inventor: Herve Bazin, Villeneuve Les Avignon (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: CIS BIO INTERNATIONAL, SACLAY (FR)

(21) Appl. No.: 12/530,527

(22) PCT Filed: Mar. 7, 2008
(86) PCT No.: PCT/FR2008/050393

§ 371 Date: Jan. 21, 2010

(30) Foreign Application Priority Data
Mar. 9, 2007 (FR) 07 53738

(51) Int. Cl. C12Q 1/02; C07F 9/04

(52) U.S. Cl. 435/29; 548/414

(57) ABSTRACT

A subject matter of the invention is cyanine derivatives of formula:

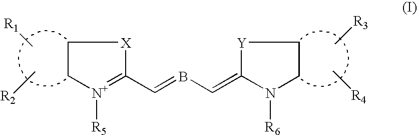

in which the dotted lines represent the atoms necessary for the formation of one or two fused aromatic rings, each ring comprising 5 or 6 carbon atoms;
R₁, R₂, R₃ and R₄ represent, independently of one another: H; substituted or unsubstituted C₁-C₁₅ alkyl; C₁-C₆ alkoxy; (C₂-C₁₂)dialkylamino; C₁-C₆ alkoxycarbonyl; di(C₂-C₁₂)alkylamido; a substituted or unsubstituted aryl, arylalkyl or aryloxy group; a halogen atom; a nitro; an L1-W, L2-M, L2-A or L2-G group;
R₅ and R₆ represent, independently of one another: substituted or unsubstituted C₁-C₁₅ alkyl; a substituted or unsubstituted aryl or arylalkyl group; an L1-W, L2-M, L2-A or L2-G group;
X is chosen from: O, S or CR₇R₈; Y is chosen from: O, S or CR₉R₁₀;
R₇, R₈, R₉ and R₁₀ independently represent: substituted or unsubstituted C₁-C₁₅ alkyl; substituted or unsubstituted aryl, arylalkyl or aryloxy; an L1-W, L2-M, L2-A or L2-G group;
R₇ and R₈ and/or R₉ and R₁₀ can also together form a ring comprising 5 or 6 atoms or a heterocycle comprising 4 to 5 carbon atoms and an oxygen atom;
B represents a polymethine bridge comprising 1 to 5 methine groups, said groups being in particular individually unsubstituted or substituted by a substituted or unsubstituted C₁-C₁₅ alkyl; a substituted or unsubstituted aryl, arylalkyl or aryloxy group; a nitro group; an L1-W, L2-M, L2-A or L2-G group;
L1 and L2 are connecting arms; G is a reactive group; A is a coupling agent; M is a conjugated molecule, W is a phosphate or phosphonate ester (preferably diester), with the proviso that the cyanine derivative comprises at least one L1-W group and at least one L2-A, L2-G or L2-M group.

### FIELD OF THE INVENTION

A subject matter of the present invention is cyanine derivatives which absorb light and emit fluorescence in the red and the near infrared. These cyanine derivatives are capable of passing through cell membranes and are functionalized so that they can be easily used as fluorescent labels.

The invention also relates to fluorescent conjugates comprising a cyanine derivative of the invention covalently bonded to a coupling agent making possible the labeling of molecules in the cell. The cyanine derivatives according to the invention are particularly suitable for the labeling of proteins in cells.

Finally, the invention also relates to fluorescent conjugates comprising a cyanine derivative of the invention covalently bonded to a substance which it is desired to insert into the cell, said substance being rendered fluorescent by the coupling with the cyanine derivative.

Another subject matter of the invention is the use of the cyanine derivatives of the invention to label products present in living cells.

The compounds according to the invention are of particular use in labeling biomolecules present in the intracellular medium without modifying the integrity of the plasma membrane, in particular without using products intended to render this membrane permeable.

Indeed, the compounds according to the invention have a lipophilic nature, insofar as they do not have sulfate, sulfonate, phosphate, phosphonate or carboxylate groups conventionally used in the prior art to solve problems of aggregation. These groups have been replaced with phosphonate or phosphate esters (preferably diesters) which do not affect the lipophilic nature of these compounds and which make it possible, after hydrolysis by intracellular enzymes, to prevent phenomena of aggregation in the cell.

The invention thus also relates to the use of cyanine derivatives according to the invention comprising a coupling agent as fluorescent labels.

The invention relates in addition to a method for labeling a biomolecule present in an intact cell which consists in introducing, into the extracellular medium, a derivative according to the invention comprising a coupling agent, said biomolecule comprising a coupling domain. This method is highly advantageous since it makes it possible to label compounds inside cells without rendering their membrane permeable.

### TECHNICAL FIELD

Fluorescent dyes which absorb and emit light in the visible and near infrared spectral region have been commonly used for various analytical applications in the biochemical, biological and diagnostic fields, in particular due to their molar absorption and/or their high fluorescence quantum yield.

In particular, only cyanine derivatives absorb and emit in the near infrared, a region of the electromagnetic spectrum of particular advantage in studying living cells as this wavelength range makes it possible to avoid the bleaching due to the photochemical decomposition of the dyes used as fluorescent labels (this phenomenon worsens as a function of the energy of the photons and is thus not very pronounced in the near infrared). Furthermore, biological molecules do not exhibit autofluorescence in the near infrared, which limits problems of nonspecific background noise.

Despite these advantages, the lipophilic nature of cyanines, due to the presence of polycyclic groups and of a hydrophobic polymethine chain, is not always compatible with a good performance as fluorescent label. For example, cyanines have a tendency to form aggregates in solution. Thus, indocyanine green experiences a fall in its quantum yield as a result of extinction of fluorescence due to the formation of aggregates when it is used at concentrations greater than 125 μM. This phenomenon, which is fairly general (it also exists in the case of fluorescein), is particularly pronounced in the cyanine series.

Furthermore, and despite this lipophilic nature, cyanines pass through biological membranes very poorly, indeed even not at all, in particular due to their high molecular weight (of the order of 700-800 daltons), whereas smaller molecules, such as fluorescein (337 daltons), do not exhibit this disadvantage.

The technical solutions for overcoming the problem of solubility and of aggregation of cyanines are not very satisfactory. Conjugates of cyanines with hydrophilic peptides, with polyethylene glycols or with oligosaccharides have been used and several studies have been devoted to the use of dyes for the near infrared and with regard to their conjugates with biomolecules. This approach has the disadvantage of requiring multistage syntheses, which implies modest yields. Moreover, in the case of the use of hydrophilic oligopeptides or oligosaccharides, the cost price may be high and the purification of these conjugates requires purification techniques other than those used in conventional organic synthesis.

The most commonly used approach for improving the photophysical properties and thus the analytical performance as fluorescent tracer has been to limit the phenomenon of aggregation by enhancing the hydrophilic nature by the introduction of chemical groups carrying a negative charge onto the indole ring systems, such as sulfonates and/or alkylsulfonate (sulfoalkyl) groups and also carboxyl groups. Thus, nonsulfonated indocyanine derivatives (such as the product known under the trade name IR-786 from Sigma) have solubilities of less than 10 μM and their use in an aqueous medium requires the use of adjuvants. The tetrasulfonation of these derivatives makes it possible to increase the solubility in an aqueous medium up to more than 10 mM. On the other hand, the sulfonation has only a slight impact on the fluorescence quantum yield, which is on average less than 15% for a tetrasulfonated compound.

### PRIOR ART

International application WO 2005/056689 A2 relates to compounds which are dyes at physiological pH, which compounds have a cyanine backbone and comprise a negatively charged substituent.

International application WO 2005/061456 A1 relates to a near infrared fluorescent contrast medium comprising a cyanine compound which exhibits a water-soluble group.

International application WO 2005/089813 A2 relates to cyanine dyes and to biological conjugates of these used in diagnostic imaging and in therapeutics. The conjugates consist in particular of several cyanine dyes with a variety of bis- and tetrakis(carboxylic acid) homologs.

International application WO 2005/056687 A2 has as subject matter cyanine dyes, the methine residue of which is substituted, which are suitable for the labeling of nucleic acids and which consist of cyanine derivatives substituted in particular by sulfoalkyl or carboxyalkyl chains.

International application WO 2004/039894 A2 relates to functionalized cyanine dyes and to their derivatives of use as intermediates in the preparation of cyanine dyes, and also to processes for the preparation of these dyes and to the dyes thus obtained. The cyanine derivatives all comprise sulfonate functional groups.

International application WO 03/082988 A1 relates to water-soluble near-infrared fluorochromes of use, for example, for biomedical imaging.

International application WO 02/068537 A2 relates to fluorescent dyes, in particular fluorescent cyanine dyes, which are soluble in water and which comprise additional sites for attaching to biomolecules. The solubility in water is provided by the presence of sulfonate groups.

International application WO 02/24815 A1 describes cyanine derivatives comprising sulfonate groups.

International application WO 01/77229 A2 relates to cyanine dyes which exhibit alkyl substituents in the meso position of the methine chain, sulfoaryl groups and at least one reactive group which makes possible binding to a target substance.

International application WO 01/52746 A1 relates to dye-peptide conjugates which are suitable for medical imaging and therapeutics. These dye-peptide conjugates comprise several cyanine-based dyes with a variety of bis- and tetrakis(carboxylic acid) homologs.

International application WO 00/66664 A1 relates to asymmetric cyanine dyes comprising a cyclic azabenzolium fragment, in particular cyanine dyes substituted by a cationic side chain, monomeric and dimeric cyanine dyes, cyanine dyes which are reactive chemically and cyanine dye conjugates.

International application WO 01/57237 A2 describes nonfluorescent cyanines comprising an NO₂ group and carboxylic acid esters.

An attempt has been made to use a phosphonate (phosphonic acid) functional group with the aim of reducing the phenomenon of aggregation of cyanines [Oswald B. et al., Novel diode laser-compatible fluorophores and their application to single molecule detection, protein labeling and fluorescence resonance energy transfer immunoassay. Photochem. Photobiol. (2001), 74(2), 237-45].

International application WO 01/36973 A2 relates to a process for improving the solubility in water of optical labels which consists in introducing phosphoric acid residues or phosphonic acid residues, or their salts of monoesters, into the labels. These labels are of use in the labeling of biomolecules, of polymers and of pharmaceutical agents. This application describes in particular a pentamethine cyanine carrying an alkylphosphonate monoester on the nitrogen of the indole ring.

Gruber et al. (Journal of Fluorescence, Vol. 15, No. 3, May 2005, 207-214) describe two cyanine-derived compounds (named chromeon 546 and chromeon 642) in which one of the nitrogen atoms is substituted by a phosphonate ethyl ester carried by a dimethylene arm and the other is substituted by an N-hydroxysuccinimide reactive group carried by a polymethylene arm. It should be noted that the phosphonate group does not make it possible, a priori, for these compounds to pass through biological membranes.

Mazières et al. (Dyes and Pigments, 74 (2007), pp 404-409) describe symmetrical cyanines substituted on the nitrogens of the indole rings by diethoxyphosphorylpropyl groups. These compounds, after hydrolysis, result in cyanines carrying water-soluble phosphonic acid groups.

Patent application US 2006/0199955 discloses cyanines comprising a zwitterionic phosphonate group intended to increase the polarity of the cyanine and thus its solubility; the compounds described also carry a sulfonate group and thus cannot pass through the cell membranes of intact cells.

Dyes which are fluorescent in the near infrared and which have a carbocyanine structure and also charged functional groups, such as those described above (sulfate, sulfonate, carboxyl, phosphate or phosphonate), are reported as exhibiting a low membrane permeability because of their high molecular weight (compared, for example, with that of rhodamine=334 Da) and by the presence of multiple charges. It has also been observed that, with the increase in the charge number, the fluorescent dyes remain in the extracellular compartments [Frangioni J. V., Curr. Opin. Chem. Biol., 2003, 7(5), 626-34. In vivo near-infrared fluorescence imaging]. Finally, when several fluorescent compounds of this type are attached to the same biomolecule, an extinction of fluorescence has been observed [Lin Y. et al., Bioconjug., Chem., 2002, 13(3), 605-10. Novel near-infrared cyanine fluorochromes: synthesis, properties and bioconjugation].

There thus exists a need for cyanine derivatives which are capable of passing through cell membranes, which do not exhibit a propensity to aggregate and which can be used as fluorescent labels. It is important to note that the solutions of the prior art for overcoming aggregation (addition of negatively charged groups) conflict with good membrane permeability. Conversely, the lipophilic cyanines which are capable of passing through the membranes have a tendency to aggregate.

The parameters governing the propensity of a dye to pass through the membranes of cells are known (lipophilic nature, polar nature and charge which, taken together, define an amphiphilic nature, size and shape of the molecule) but their relative influence and their use as predictive tool for the property of passing through membranes remains limited.

Despite these obstacles, novel cyanine derivatives have now been developed which are capable of passing through cell membranes without, however, exhibiting problems of aggregation and of solubility.

The cyanines according to the invention have the distinguishing feature of comprising:
at least one phosphate ester (preferably diester) or one phosphonate ester (preferably diester) grafted to the cyanine via a connecting arm, and
a functional group, or else a coupling agent, or also a conjugated substance which it is desired to insert into the cell in the labeled form, this functional group, coupling agent or substance being carried by a connecting arm.

The compounds according to the invention are capable of passing through cell membranes, insofar as they comprise hydrophobic groups specific to cyanines (polycycles, polymethine chains) and do not comprise polar groups, such as those provided in the prior art for overcoming the problem of aggregation and of solubility: the compounds according to the invention do not comprise sulfate, sulfonate, phosphate, phosphonate or carboxylate groups.

On the other hand, the cyanine derivatives according to the invention comprise at least one phosphate or phosphonate ester (preferably diester): these groups have the distinguishing feature of undergoing hydrolysis in the cytoplasm of the cell, which hydrolysis is catalyzed by cell enzymes, in particular phosphodiesterases, this being the case despite the presence of the cyanine structure, the size of which might have interfered with the enzymatic processes.

Furthermore, the cyanine derivatives according to the invention are functionalized, which allows a person skilled in the art to couple them to any group or molecule which he desires to label. The products according to the invention can also comprise a coupling agent which will allow them to label biomolecules comprising an appropriate coupling domain.

These characteristics make the cyanine derivatives according to the invention particularly suitable compounds for the labeling of intracellular biological molecules by fluorescent compounds which can be excited and which emit in the red and the near infrared, without necessarily rendering the cells permeable.

### DESCRIPTION OF THE INVENTION

The fluorescent compounds according to the invention are cyanine derivatives of formula (I):

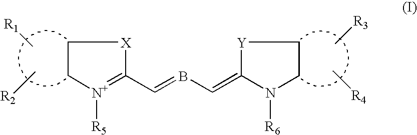

in which the dotted lines represent the atoms necessary for the formation of one or two fused aromatic rings, each ring comprising 5 or 6 carbon atoms;
R₁, R₂, R₃ and R₄ represent, independently of one another:
   a hydrogen atom;
   a substituted or unsubstituted C₁-C₁₅ alkyl group;
   a C₁-C₆ alkoxy group;
   a (C₂-C₁₂) dialkylamino group;
   a C₁-C₆ alkoxycarbonyl group;
   a (C₂-C₁₂) dialkylamino group;
   a substituted or unsubstituted aryl, arylalkyl or aryloxy group;
   a halogen atom;
   a nitro group;
   a group chosen from: L1-W, L2-M, L2-A or L2-G;
R₅ and R₆ represent, independently of one another:
   a substituted or unsubstituted C₁-C₁₅ alkyl group;
   a substituted or unsubstituted aryl or arylalkyl group;
   a group chosen from: L1-W, L2-M, L2-A or L2-G;
X is chosen from: O, S or CR₇R₈;
Y is chosen from: O, S or CR₉R₁₀;
R₇, R₈, R₉ and R₁₀ independently represent:
   a substituted or unsubstituted C₁-C₁₅ alkyl group;
   a substituted or unsubstituted aryl, arylalkyl or aryloxy group;
   a group chosen from: L1-W, L2-M, L2-A or L2-G;
R₇ and R₈ and/or R₉ and R₁₀ can also together form a ring comprising 5 or 6 atoms or a heterocycle comprising 4 to 5 carbon atoms and an oxygen atom;
B represents a polymethine bridge comprising 1 to 5 methines, in which the methine groups are individually unsubstituted or substituted by a group chosen from:
   a substituted or unsubstituted C₁-C₁₅ alkyl group;
   a substituted or unsubstituted aryl, arylalkyl or aryloxy group;
   a nitro group;
   a group chosen from: L1-W, L2-M, L2-A or L2-G;
or else two substituents of adjacent methines can together form a saturated or unsaturated hydrocarbon ring comprising 4, 5 or 6 atoms which is optionally substituted one or more times by a group chosen from:
   a substituted or unsubstituted C₁-C₁₅ alkyl group;
   a substituted or unsubstituted aryl, arylalkyl or aryloxy group;
   a halogen atom;
   a nitro group;
   a group chosen from: L1-W, L2-M, L2-A or L2-G;
L1 and L2 are connecting arms;
G is a reactive group;
A is a coupling agent;
M is a conjugated molecule;
W is a phosphate or phosphonate ester (preferably diester) as defined below:
   with the proviso that the cyanine derivative comprises one or two L1-W groups and one or two groups chosen from L2-A, L2-G and L2-M.

The cyanine derivatives of formula (I) of the invention comprise a counterion “Z” (not represented) which counterbalances the positive or negative charge or charges of the cyanine derivative.

The nature of the counterion is not essential according to the invention; it depends on the synthesis process employed or on the medium in which the cyanine derivatives occur.

For example, Z will be:
I⁻ in the case of an alkylation by an iodide, such as, for example, CH₃CH₃I;
Cl⁻ if a hydrolysis is carried out with HCl;
CF₃COO⁻ or HCOO⁻ in the case of purification by reverse phase HPLC in the presence respectively of trifluoroacetic acid (CF₃COOH) or of formic acid (HCOOH).

Advantageously, the counterion Z is chosen from I⁻, Cl⁻, Br⁻, CF₃COO⁻, HCOO⁻ or Na⁺.

### DEFINITIONS

In the present description, the groups have the following meanings:
C₁-C₁₅ alkyl group: linear, branched or cyclic (in this case, optionally interrupted by a heteroatom, such as O, S or N) hydrocarbon chain comprising from 1 to 15 carbon atoms and optionally substituted by one or more substituents chosen from the following groups: chloro, fluoro, bromo, nitro, C₁-C₆ alkoxy, C₂-C₁₂ dialkylamino, C₁-C₆ alkoxycarbonyl, C₁-C₆ alkylcarboxylate, N,N—(C₂-C₁₂) dialkylamido or amido. Examples of alkyl groups are methyl, ethyl, isopropyl, n-propyl, butyl, tert-butyl, n-hexyl, n-decyl, n-dodecyl, cyclohexyl or octyl.
C₁-C₆ alkoxy group: linear or branched hydrocarbon chain comprising from 1 to 6 carbon atoms bonded to an oxygen atom. Examples of alkoxy groups are the following groups: methoxy, ethoxy, propoxy, tert-butoxy and n-butoxy.
C₁-C₆ alkoxycarbonyl group: linear or branched hydrocarbon chain comprising from 1 to 6 carbon atoms bonded to the oxygen of a carboxyl group —OOC—.
C₁-C₆ alkylcarboxyl group: linear or branched hydrocarbon chain comprising from 1 to 6 carbon atoms bonded to the carbon of a carboxyl group —COO—.
(C₂-C₁₂) dialkylamino group: nitrogen comprising two linear or branched alkyl groups which are identical or different and which are each composed of 2 to 12 carbon atoms.
N,N—(C₂-C₁₂) dialkylamido group: (C₂-C₁₂)dialkylamino group bonded to the carbon of a carbonyl group —CO—.
C₅-C₁₄ aryl group: aromatic ring comprising 5 or 6 carbon atoms or aromatic bicycle comprising 8 to 10 atoms or aromatic tricycle comprising 10-14 atoms. The aryl group can optionally be substituted by one or more substituents chosen from the following groups: C₁-C₁₅ alkyl, chloro, fluoro, bromo, nitro, C₁-C₆ alkoxy, di(C₁-C₁₅)alkylamino or C₁-C₆ alkoxycarbonyl.
heteroaryl group comprising 5-10 elements: aryl group having a ring or rings comprising from 5 to 10 carbon atoms, at least one of which is replaced by a heteroatom chosen from N, O or S. A heteroaryl group can optionally be substituted by one or more substituents chosen from the following groups: C₁-C₁₅ alkyl, chloro, fluoro, bromo, nitro, C₁-C₆ alkoxy, di(C₁-C₁₅)alkylamino or C₁-C₆ alkoxycarbonyl. Examples of heteroaryl groups are pyrrolyl, pyridyl, thienyl, furanyl, oxazolyl, isoxazolyl, oxadiazolyl, imidazolyl, benzoxazolyl, benzimidazolyl, quinolyl, benzofuranyl, indolyl, carbazolyl, coumarinyl and benzocoumarinyl.
C₆-C₂₅ arylalkyl group: C₅-C₁₀ aryl group bonded to a C₁-C₁₅ alkyl group.
a halogen: chloro-, fluoro-, iodo- or bromo-.
connecting arm B: can be a single covalent bond or a spacing arm comprising from 1 to 20 atoms other than hydrogen chosen from carbon, nitrogen, phosphorus, oxygen and sulfur atoms, this connecting group being linear or branched, cyclic or heterocyclic and saturated or unsaturated and composed of a combination of bonds chosen from: carbon-carbon bonds which can be single, double, triple or aromatic; carbon-nitrogen bonds; nitrogen-nitrogen bonds; carbon-oxygen bonds; carbon-sulfur bonds; phosphorus-oxygen bonds; phosphorus-nitrogen bonds; ether bonds; ester bonds; thioether bonds; amine bonds; amide bonds; carboxamide bonds; sulfonamide bonds; urea bonds; urethane bonds; hydrazine bonds; or carbamoyl bonds.
intact cell: denotes a cell, the membrane integrity and the intracellular integrity of which are retained, for example a cell which has not been submitted to a chemical treatment intended to render its membrane permeable.

### PREFERRED COMPOUNDS OF THE INVENTION

The preferred cyanine derivatives according to the invention are the compounds corresponding to the following formulae, in which the R₁—R₆, B, X and Y groups are as defined above:

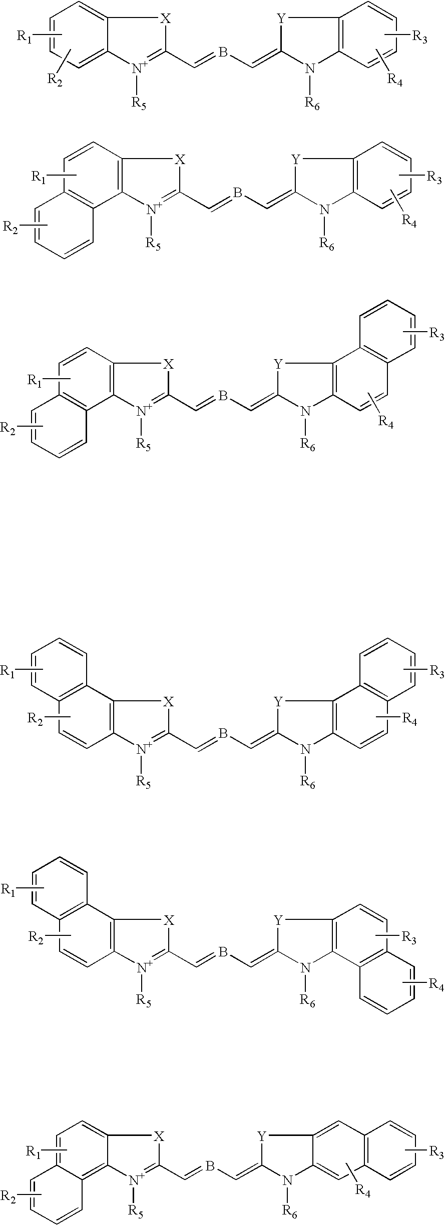

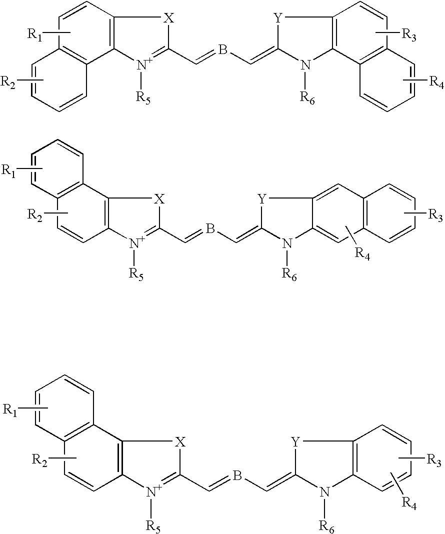

### The Polymethine Bridge B

The compounds of the invention comprise a polymethine bridge between the cyclic structures. As indicated above, these methines can be substituted or adjacent methines can together form a saturated or unsaturated hydrocarbon ring comprising 4, 5 or 6 elements which is optionally substituted.

The preferred compounds of the invention comprise unsubstituted methines, or else only the central methine is substituted, or also two central methines form a ring.

By way of illustration and without implied limitation, the bridge can be composed of the following polymethine chains:

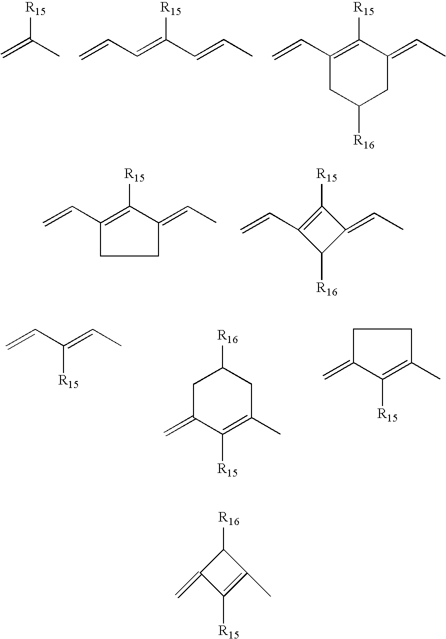

in which R₁₅ and R₁₆ are chosen from:
a hydrogen atom;
a substituted or unsubstituted C₁-C₁₅ alkyl group;
a substituted or unsubstituted aryl, arylalkyl or aryloxy group;
a halogen atom;
a nitro group;
a group chosen from: L2-M, L2-A, L2-G or L1-W.
   L1 and L2 are connecting arms,
   G is a reactive group,
   A is a coupling agent,
   M is conjugated molecule,
   W is a phosphate or phosphonate ester (preferably diester).

Preferably, the polymethine bridge corresponds to one of the formulae below:

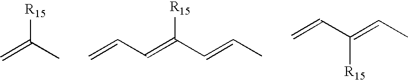

### The Phosphate or Phosphonate Ester (Preferably Diester)

The phosphate or phosphonate esters (preferably diesters) carried by an arm which are present on the cyanine derivatives according to the invention offer several technical advantages: they do not increase the polarity of the compounds and thus promote their passage through lipid biological membranes; moreover, once inside the cell, the phosphate or phosphonate esters (preferably diesters) are hydrolyzed by cell enzymes to give phosphate or phosphonate groups. These phosphate or phosphonate groups contribute to the effectiveness of the cyanines according to the invention in labeling in particular proteins, since they make it possible to overcome the phenomena of aggregation generally observed when cyanines are used in this application.

These groups can be grafted to the cyanine according to synthesis methods known to a person skilled in the art. For example, the introduction of a phosphonate ester onto an aromatic ring system is carried out starting from a brominated derivative, such as 5-bromo-2,3,3-trimethyl-3H-indole, as described in example 13, by following the protocol described in the literature [A Novel Synthesis of Dialkyl Arenephosphonates, Hirao T. et al., Synthesis, (1), 56-57 (1981)]. The experimental part below illustrates routes for the synthesis of cyanine derivatives comprising phosphate or phosphonate esters.

In the case where phosphate esters are introduced onto aromatic rings of the cyanine, these groups necessarily have to be carried by a connecting arm.

The phosphate or phosphonate ester (preferably diester) groups W have the formulae:

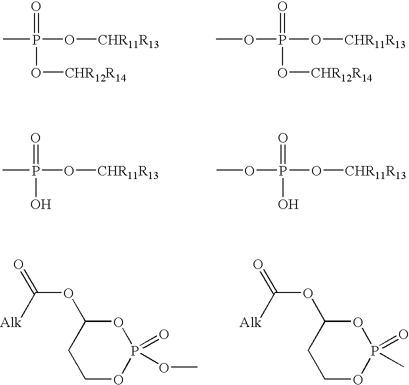

in which:
R₁₁ and R₁₂ are identical or different and are chosen from:
   a hydrogen atom;
   an unsubstituted C₁-C₅ alkyl group;
R₁₃ and R₁₄ are identical or different and are chosen from:
   a hydrogen atom;
   an unsubstituted C₁-C₁₅ alkyl group;
   a C₁-C₆ alkoxycarbonyl group;
   a C₁-C₆ alkylcarboxyl group;
   an N,N—(C₂-C₁₂)dialkylamido group;
   an amido group;
   a group of formula —C—S—CO-Alk,
Alk being an unsubstituted linear or branched C₁-C₄ alkyl;
R₁₁ and R₁₂ and/or R₁₃ and R₁₄ can also together form a phthalidyl group of formula:

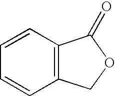

The cyanine derivatives which are particularly preferred according to the invention are those in which the phosphate/phosphonate esters are grafted to the indole aromatic ring of the cyanine.

The phosphonate diesters with the following general formula are preferred:

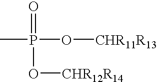

in which:
R₁₁ and R₁₂ are identical and are chosen from:
   a hydrogen atom;
   an unsubstituted C₁-C₅ alkyl group;
R₁₃ and R₁₄ are identical and are chosen from the following groups:
   methylcarboxyl(=—O—CO—CH₃=acetoxymethyl ester);
   tert-butylcarboxyl(=—O—CO—C(CH₃)₃=trimethyl-acetoxymethyl ester);
   methyloxycarbonyl(=—CO—OCH₃=methyl glycolate ester);
   methanamido(=—CO—NH₂=glycolamide ester);
   N,N-dimethylmethanamido(=—CO—N(CH₃)₂=substituted glycolamide ester);
   N-methylmethanamido.

In particular, the cyanine derivatives comprising the following phosphonate esters are more easily hydrolyzed by cell enzymes than simple alkyl esters:

### Acetoxymethyl Ester

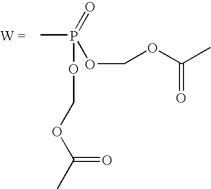

Acetoxymethyl ester, the methyl group optionally being substituted by an H or C₁-C₅ alkyl group:

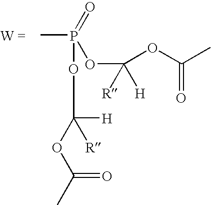

### Trimethylacetoxymethyl Ester:

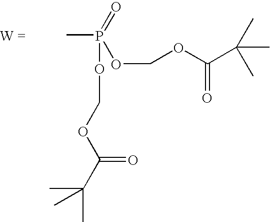

Methyl glycolate or ethyl glycolate ester (glycolic acid ═HOCH₂COOH, methyl glycolate ═HOCH₂COOMe)

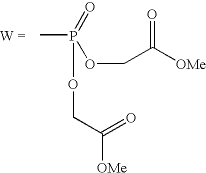

Unsubstituted glycolamide ester (glycolamide=HOCH₂CONH₂)

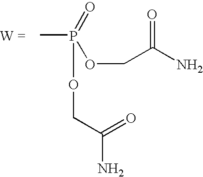

Substituted glycolamide ester (N-methylglycolamide=HOCH₂CONHCH₃ or N,N-dimethylglycolamide):

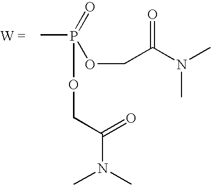

Preference is very particularly given, among the compounds of formula (I) above or the preferred compounds of the invention, to:

1) the compounds in which:
X is the CR₇R₈ group and/or Y is the CR₉R₁₀ group;
one or two R₁—R₄ groups represent an L1-W group; and
one or two R₇—R₁₀ groups represent L2-A, L2-G or L2-M;
   2) the compounds in which:
X is the CR₇R₈ group and/or Y is the CR₉R₁₀ group;
R₅ and/or R₆ represent an L1-W group; and
one or two R₇—R₁₀ groups represent an L2-A, L2-G or L2-M group;
   3) the compounds in which:
R₅ and/or R₆ represent an L1-W group;
R₁₅ or R₁₆ of the polymethine bridge B represents an L2-A, L2-G or L2-M group;
   4) the compounds in which:
one or two R₁—R₄ groups represent an L1-W group; and
R₁₅ or R₁₆ of the polymethine bridge B represents an L2-A, L2-G or L2-M group.

A family of particularly preferred compounds of the invention is that composed of the compounds of formula:

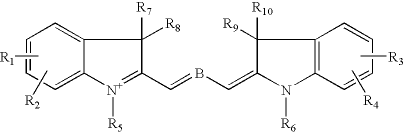

in which:
R₁ and R₃ are hydrogen atoms;
R₇, R₈ and R₉ independently represent a substituted or unsubstituted C₁-C₁₅ alkyl group;
R₅ and R₆ represent, independently of one another, a substituted or unsubstituted C₁-C₁₅ alkyl group;
B represents a polymethine bridge comprising from 1 to 5 unsubstituted methines;
R₁₀ represents a group chosen from L2-M, L2-A or L2-G;
   L2 is a connecting arm;
   G is a reactive group;
   A is a coupling agent;
   M is a conjugated molecule;
R₂ and R₄ are identical and represent an L1-W group;
   L1 is a connecting arm chosen from: a single bond or a group of formula —(CH₂)ₙ—, n being an integer between 2 and 8;
   W is a phosphonate diester chosen from the groups with the following formulae:

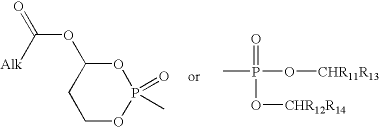

in which:
R_{H} and R₁₂ are identical or different and are chosen from:
   a hydrogen atom;
   an unsubstituted C₁-C₅ alkyl group;
R₁₃ and R₁₄ are identical or different and are chosen from:
   a hydrogen atom;
   an unsubstituted C₁-C₁₅ alkyl group;
   a C₁-C₆ alkoxycarbonyl group;
   a C₁-C₆ alkylcarboxyl group;
   an N,N—(C₂-C₁₂)dialkylamido group;
   an amido group;
   a group of formula —C—S—CO-Alk, Alk being an unsubstituted linear or branched C₁-C₄ alkyl;
R₁₁ and R₁₂ and/or R₁₃ and R₁₄ can also together form a phthalidyl group of formula:

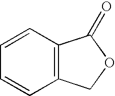

Another family of particularly preferred compounds of the invention is that composed of the compounds of formula:

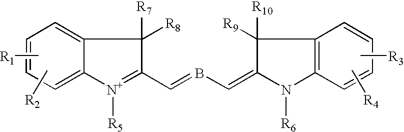

in which:
R₁, R₂, R₃ and R₄ are hydrogen atoms;
R₇, R₈ and R₉ independently represent a substituted or unsubstituted C₁-C₁₅ alkyl group;
B represents a polymethine bridge comprising from 1 to 5 unsubstituted methines;
R₁₀ represents a group chosen from L2-M, L2-A or L2-G;
   L2 is a connecting arm;
   G is a reactive group;
   A is a coupling agent;
   M is a conjugated molecule;
R₅ and R₆ are identical and represent an L1-W group;
   L1 is a connecting arm chosen from a single bond or a group of formula —(CH₂)ₙ—, n being an integer between 2 and 8;
   W is a phosphonate diester with a formula chosen from the following formulae:

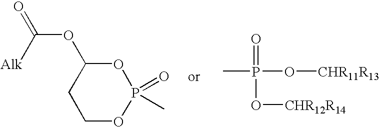

in which:
R₁₁ and R₁₂ are identical or different and are chosen from:
   a hydrogen atom;
   an unsubstituted C₁-C₅ alkyl group;
R₁₃ and R₁₄ are identical or different and are chosen from:
   a hydrogen atom;
   an unsubstituted C₁-C₁₅ alkyl group;
   a C₁-C₆ alkoxycarbonyl group;
   a C₁-C₆ alkylcarboxyl group;
   an N,N—(C₂-C₁₂)dialkylamido group;
   an amido group;
   a group of formula —C—S—CO-Alk, Alk being an unsubstituted linear or branched C₁-C₄ alkyl;
R₁₁ and R₁₂ and/or R₁₃ and R₁₄ can also together form a phthalidyl group of formula:

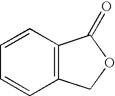

### Coupling Agent A

The cyanine derivatives according to the invention can comprise a coupling agent carried by a connecting arm. Preferably, this coupling agent is capable of passing through the cell membrane.

The role of this coupling agent is to make possible the covalent or noncovalent coupling of the cyanine with a target molecule, for example present in a cell. For this purpose, the target molecule must comprise the appropriate coupling domain. In the case where the target molecule is present in a cell, it is necessary for the coupling agent to be itself capable of passing through the plasma membrane.

The coupling agent A can thus be an antibody, an antibody fragment or a peptide aptamer specific for the target molecule. These antibodies, antibody fragments or aptamers can recognize a domain naturally present in the target molecule or also a domain introduced into this molecule by techniques well known to a person skilled in the art, in particular molecular biology techniques which make possible the introduction into a biomolecule of a protein tag.

The coupling agent and domain can be members of a pair of binding partners, the two members being capable of binding noncovalently, such as, for example, the following pairs:
avidin (or streptavidin)/biotin: this pair can be used to label a protein of interest expressed by the cell in the form of a fusion protein with the binding domain of avidin or streptavidin. In this case, the cyanine derivatives according to the invention are conjugated to biotin as coupling agent. The (strept) avidin/biotin pair is known to a person skilled in the art, who will have no difficulty in coupling the cyanine derivatives according to the invention with biotin.
biarsenic/tetracysteine unit compounds: this pair of binding partners was first described by Adams et al. (“New biarsenical ligands and tetracysteine motifs for protein labeling in vitro and in vivo: synthesis and biological applications”, J. Am. Chem. Soc., 2002 May 29, 124(21), 6063-76). A cyanine derivative according to the invention, conjugated to a biarsenic unit, makes it possible to label a protein of interest expressed by the cell in the form of a fusion protein with a tetracysteine domain capable of binding with the biarsenic unit. The sequence of the tetracysteine domain is Cys-Cys-aa1-aa2-Cys-Cys (in which aa1 and aa2 are any natural amino acid and preferably aa1=Pro and aa2=Gly).
methotrexate (or trimethoprim)/dihydrofolate reductase (DHFR): high affinity binding between DHFR and compounds, such as methotrexate or trimethoprim, is described in particular by Miller et al. (“Methotrexate conjugates: a molecular in vivo protein tag”, Angew. Chem. Int. Edn. Engl., 43, 1672-1675 (2004)). Cyanine derivatives according to the invention, conjugated to methotrexate or to trimethoprim as coupling agent, will be capable of labeling a protein of interest expressed by the cell in the form of fusion protein with DHFR.
SLF′/FKBP (F36V): Clackson et al. have described the pair composed of an artificial ligand SLF′ which binds to a mutant of the protein FKBP (“Redesigning an FKBP-ligand interface to generate chemical dimerizers with novel specificity”, Proc. Natl. Acad. Sci. USA, 1998 Sep. 1, 95(18), 10437-42). Cyanine derivatives according to the invention, conjugated to SLF′ as coupling agent, will be capable of labeling a protein of interest expressed by the cell in the form of a fusion protein with FKBP (F36V).

The coupling agent A of the cyanine derivatives according to the invention can also be an agent which makes coupling possible via covalent bonding with a coupling domain present on the molecule of interest which it is desired to label.

In particular, the coupling agent can be the substrate for a “suicide” enzyme present in the cell. In this case, the coupling domain is thus a suicide enzyme and is expressed by the cell in the form of a fusion protein composed of the suicide enzyme and of the protein of interest which it is desired to label. Suicide enzymes are proteins which have an enzymatic activity modified by specific mutations which confer on them an ability to rapidly and covalently bind a substrate. These enzymes are said to be “suicide” enzymes as each can bind only a single fluorescent molecule, the activity enzyme being blocked by the attaching of the substrate.

Suicide enzymes have recently been used in methods for labeling proteins: these methods consist in manufacturing a fusion protein, comprising a protein which it is desired to label and a suicide enzyme, and in bringing this fusion protein into contact with, for example, a labeling fluorophore covalently bonded to the substrate for said suicide enzyme.

Currently, two known families of suicide enzymes make this type of labeling possible:
the mutant of an alkylguanine-DNA alkyltransferase (or “SnapTag” sold by Covalys, described in application WO 02/083937 A2), the substrate for which is benzylguanine or a benzylguanine derivative. Benzylguanine derivative is understood to mean a benzylguanine which is modified but which is nevertheless recognized by the suicide enzyme. Such substrates are described in applications WO 2005/085470 and WO 2004/031405;
the mutant of a haloalkane dehalogenase (“HaloTag” sold by Promega) which also generates an enzymatic reaction of the suicide type (technique described in WO 2004/072232 A2), the substrate for which is a haloalkane, preferably a chloroalkane. Substrates are described in WO 2004/072232 and WO 2006/093529.

In the case where it is desired to label such suicide enzymes with a cyanine derivative according to the invention, said cyanine will comprise the coupling agent, which will be a benzylguanine group (or one of its derivatives) or also a haloalkane (preferably a chloroalkane), bonded to the cyanine via a connecting arm.

To summarize, the coupling agent A of the cyanine derivatives according to the invention can be a member of a pair of noncovalent binding partners or a member of a pair of covalent binding partners.

In particular, A can be chosen from the following compounds: an antibody, an antibody fragment, a peptide aptamer, biotin, a biarsenic compound, trimetroprine, methotrexate, SLF′, a benzylguanine group or a chloroalkane.

### Reactive Group G

The cyanine derivatives according to the invention can comprise a reactive group G, which is a group capable of reacting with a functional group present on another substance or molecule to form a covalent bond. In this case, the reactive group G will react with a functional group present on the substance or molecule which it is desired to conjugate to the cyanine derivative according to the invention.

Typically, the reactive group is an electrophilic or nucleophilic group which can form a covalent bond when it is brought together with an appropriate nucleophilic or electrophilic group respectively. The coupling reaction between the cyanine derivative comprising a reactive group and a molecule to be conjugated M carrying a functional group results in the formation of a covalent bond comprising one or more atoms of the reactive group. By way of examples, the pairs of electrophilic/nucleophilic groups and the type of covalent bond formed when they are brought together are listed below:

| Electrophilic | Nucleophilic | |
|---|---|---|
| group | group | Type of bond |
| acrylamides | thiols | thioethers |
| acyl halides | amines/anilines | carboxamides |
| aldehydes | amines/anilines | imines |
| aldehydes or | hydrazines | hydrazones |
| ketones | | |
| aldehydes or | hydroxylamines | oximes |
| ketones | | |
| alkyl sulfonates | thiols | thioethers |
| anhydrides | amines/anilines | carboxamides |
| aryl halides | thiols | thiophenols |
| aryl halides | amines | arylamines |
| aziridines | thiols | thioethers |
| carbodiimides | carboxylic acids | N-acylureas or |
| | | anhydrides |
| activated esters* | amines/anilines | carboxamides |
| haloacetamides | thiols | thioethers |
| halotriazines | amines/anilines | aminotriazines |
| imido esters | amines/anilines | amidines |
| isocyanates | amines/anilines | ureas |
| isothiocyanates | amines/anilines | thioureas |
| maleimides | thiols | thioethers |
| sulfonate esters | amines/anilines | alkylamines |
| sulfonyl halides | amines/anilines | sulfonamides |

*activated ester is understood to mean groups of formula COY, where Y is
a leaving group chosen from succinimidyloxy (—C₄H₄O₂) or sulfosuccinimidyloxy (—OC₄H₃O₂—SO₃H) groups;
an aryloxy group which is unsubstituted or substituted by at least one electrophilic substituent, such as nitro, fluoro, chloro, cyano or trifluoromethyl groups, thus forming an activated aryl ester;
a carboxylic acid activated by a carbodiimide group, forming an anhydride —OCORₐ or —OCNRₐNHR_{b}, in which Rₐ and R_{b} are identical or different and are chosen from C₁-C₆ alkyl, C₁-C₆ perfluoroalkyl, C₁-C₆ alkoxy or cyclohexyl groups;
3-dimethylaminopropyl or N-morpholinoethyl.

As nonlimiting example, the substance or molecule to be conjugated M comprises at least one of the following functional groups with which the reactive group G will react: amines, amides, thiols, aldehydes, ketones, hydrazines, hydroxylamines, secondary amines, halides, epoxides, carboxylylate esters, carboxylic acids, groups comprising double bonds or a combination of these functional groups.

The functional group carried by the molecule M which will react with the reactive group G will, for example, be an amine, thiol, alcohol, aldehyde or ketone group. Preferably, the reactive group G will react with an amine or thiol functional group.

Methods for introducing these functional groups are described in particular in C. Kessler, Nonisotopic Probing, Blotting and Sequencing, 2^{nd} edition, L. J. Kricka (1995), published by Academic Press Ltd., London, pp. 66-72.

Preferably, the reactive group G is a group derived from one of the compounds below: an acrylamide, an activated amine (for example, a cadaverine or an ethylenediamine), an activated ester, an aldehyde, an alkylhalide, an anhydride, an aniline, an azide, an aziridine, a carboxylic acid, a diazoalkane, a haloacetamide, a halotriazine, such as monochlorotriazine, dichlorotriazine, a hydrazine (including hydrazides), an imido ester, an isocyanate, an isothiocyanate, a maleimide, a sulfonyl halide, or a thiol, a ketone, an amine, an acid halide, a hydroxysuccinimidyl ester, a hydroxysulfosuccinimidyl ester, an azidonitrophenyl, an azidophenyl, a 3-(2-pyridyldithio)propionamide or glyoxal, and in particular the groups of formula:

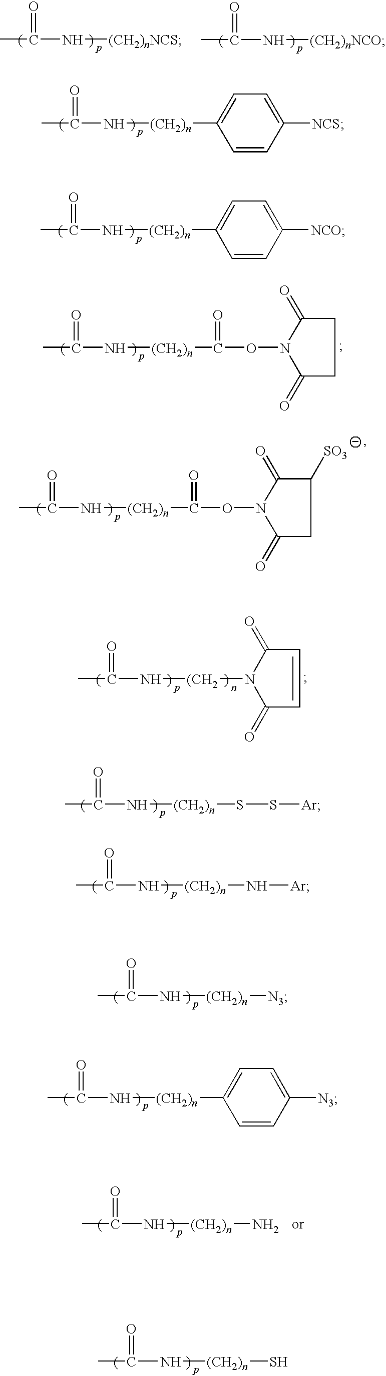

where n varies from 0 to 8 and p is equal to 0 or 1, and Ar is a 5- or 6-membered heterocycle comprising from 1 to 3 heteroatoms which is optionally substituted by a halogen atom.

Preferably, the reactive group G is a carboxylic acid, a carboxylic acid succinimidyl ester, a haloacetamide, a hydrazine, an isothiocyanate, a maleimide group or an aliphatic amine.

### Conjugated Molecule M

Although the cyanine derivatives according to the invention are particularly advantageous in the fluorescent labeling of compounds occurring in the cell, they can be coupled to any type of molecule by conventional coupling techniques and the use of reactive groups as described below.

The conjugated molecule M can be, for example, a biomolecule. Biomolecule is understood to mean a molecule present in a living organism and in particular the molecules constituting the structure of an organism and those involved in the production and conversion of energy or in the transmission of biological signals. This definition encompasses nucleic acids, proteins, sugars, lipids, peptides, oligonucleotides, metabolic intermediates, enzymes, hormones and neurotransmitters.

### Connecting Arm

The cyanine derivatives according to the invention comprise a reactive group G, a coupling agent A, a conjugated molecule M or a phosphate or phosphonate ester (preferably diester) W. Each of these groups is connected to the cyanine via a connecting arm.

In the case where cyanine comprises several of these groups, for example 2 phosphonate groups and a connecting agent, these groups can be carried by identical or different arms.

The connecting arm L which carries the G, M, A or W groups can be a single covalent bond or a spacing arm comprising from 1 to 20 atoms other than hydrogen chosen from carbon, nitrogen, phosphorus, oxygen and sulfur atoms, this connecting arm being linear or branched, cyclic or heterocyclic and saturated or unsaturated and composed of a combination of bonds chosen from: carbon-carbon bonds which can be single, double, triple or aromatic; carbon-nitrogen bonds; nitrogen-nitrogen bonds; carbon-oxygen bonds; carbon-sulfur bonds; phosphorus-oxygen bonds; phosphorus-nitrogen bonds; ether bonds; ester bonds; thioether bonds; amine bonds; amide bonds; carboxamide bonds; sulfonamide bonds; urea bonds; urethane bonds; hydrazine bonds; or carbamoyl bonds.

Preferably, the connecting arm L comprises from 1 to 20 atoms other than hydrogen which are chosen from C, N, O, P and S and can comprise combinations of ether, thioether, carboxamide, sulfonamide, hydrazine, amine or ester bonds and aromatic or heteroaromatic bonds.

Preferably, L is composed of a combination of carbon-carbon single bonds and of carboxamide or thioether bonds.

By way of example, L can be chosen from the following chains: polymethylene, arylene, alkylarylene, arylenealkyl or arylthio.

According to an advantageous aspect, the connecting arm L is composed of a divalent organic radical chosen from linear or branched C₁-C₂₀ alkylene groups optionally comprising one or more double bonds or triple bonds and/or optionally comprising one or more heteroatoms, such as oxygen, nitrogen, sulfur or phosphorus, or one or more carbamoyl or carboxamido group(s); C₅-C₈ cycloalkylene groups and C₆-C₁₄ arylene groups, said alkylene, cycloalkylene or arylene groups being optionally substituted by alkyl, aryl or sulfonate groups.

In particular, the connecting group L is chosen from the following groups:

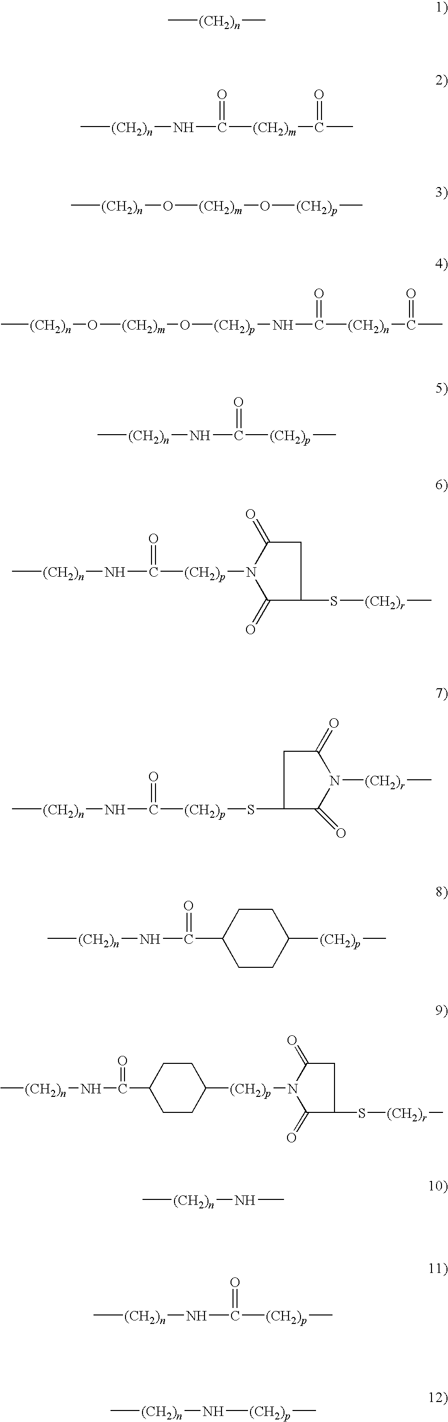

in which:
n and m are integers from 2 to 16, preferably from 2 to 8;
p and r are integers from 1 to 16, preferably from 1 to 5.

The connecting groups 1) to 9) are particularly appropriate when the reactive group is a carbamoyl group and the functional group on the substance or molecule M is an amide or thioether group.

The connecting groups 2), 6), 7) and 10) to 12) are particularly appropriate when the reactive group is an ether group and the functional group on the substance or molecule M is an amide or thioether group.

Preference is very particularly given, among the compounds of the invention, to one of the groups of compounds below:
compounds of formula (I) in which X is the CR₇R₈ group and/or Y is the CR₉R₁₀ group, one or two R₁—R₄ groups represent an L1-W group and one or two R₇—R₁₀ groups represent L2-A, L2-G or L2-M;
compounds of formula (I) in which X is the CR₇R₈ group and/or Y is the CR₉R₁₀ group, R₅ and/or R₆ represent an L1-W group and one or two R₇—R₁₀ groups represent an L2-A, L2-G or L2-M group;
compounds of formula (I) in which R₅ and/or R₆ represent an L1-W group and the polymethine bridge B is chosen from the above formulae in which R₁₅ or R₁₆ represents an L2-A, L2-G or L2-M group;
compounds of formula (I) in which one or two R₁—R₄ groups represent an L1-W group and the polymethine bridge B is chosen from the above formulae in which R₁₅ or R₁₆ represents an L2-A, L2-G or L2-M group.

Preference is very particularly given, among these compounds, to those in which the dotted lines represent the phenyl group and in which the polymethine bridge corresponds to one of the formulae below:

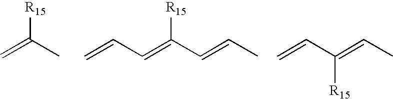

### Synthesis Processes

The synthesis of cyanine derivatives is widely described in the literature and a person skilled in the art may refer thereto in order to prepare the derivatives according to the invention. Cyanine synthesis is generally based on the use of 3 starting reactants: the polycyclic groups and the methine chain. These 3 reactants are modified before or after their condensation in order to make them carry the desired substituents.

Information on the synthesis of the intermediates used in the manufacture of the cyanines of the prior art is available in particular in the following publications:
Mujumdar et al. (1993), “Cyanine dye labeling reagents: sulfoindocyanine succinimidyl esters”, Bioconjug. Chem., 4(2), 105-11.
Mujumdar et al. (1996), “Cyanine-Labeling Reagents: Sulfobenzindocyanine Succinimidyl Esters”, Bioconjugate Chem., 7(3), 356-362. These papers allow a person skilled in the art to synthesize the intermediates necessary for the synthesis of the cyanines.
Hung, S.-C. et al. (1996), “Cyanine Dyes with High Absorption Cross Section as Donor Chromophores in Energy Transfer Primers”, Analytical Biochemistry, 243(1), 15-27.

The examples of the experimental part furthermore illustrate the synthesis of a few derivatives according to the invention.

### EXAMPLES

In these examples, the abbreviations below are used:
DIPEA: diisopropylethylamine
TSTU: N,N,N′,N′-tetramethylsuccinimidouronium tetrafluoroborate
TBTU: 2-(1H-benzotriazol-1-yl)-1,1,3,3-tetra-methyluronium tetrafluoroborate
HPLC: high performance liquid chromatography
RP-HPLC: reverse phase high performance liquid chromatography
TFA: trifluoroacetic acid
MS: mass spectroscopy
DMF: dimethylformamide
t_{R}: retention time
DY647: fluorophore sold by Dyomics, which is soluble in water, methanol and DMSO and which has similar spectral properties to those of Cy5
DY647-NHS: N-hydroxysuccinimide derivative of DY647
DMEM: acronym for the culture medium known as Dulbecco's modified essential medium
DAPI: 4′,6-diamidino-2-phenylindole dye for ring systems
ACN: acetonitrile
NHS: N-hydroxysuccinimide
BG: benzylguanine
BG-CY5: benzylguanine-cyanine CY5 conjugate
CY5: indocyanine having a polymethine bridge comprising five methines, sold by Amersham Pharmacia Biotech.
BG-DY647: benzylguanine-DY647 conjugate

### Example 1

### Synthesis of 1-[2-(diethoxyphosphoryl)-ethyl]-2,3,3-trimethyl-3H-indolium bromide

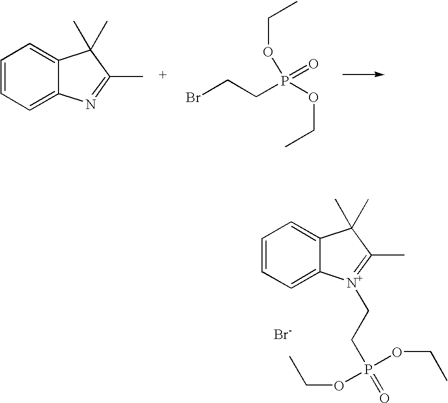

1.59 g (10.0 mmol) of 2,3,3-trimethyl-3H-indole and 2.94 g (12.0 mmol) of diethyl bromoethylphosphonate are mixed and heated at 70° C. for 40 h with stirring. After cooling the reaction mixture, 10 ml of methanol are added and then, subsequently, 30 ml of water. The desired compound is isolated by column chromatography (silica RP-18) with a methanol/water gradient ranging from 1:1 to 5:1. MS (ESI⁺): 324.2. Yield: 775 mg (19%).

### Example 2

### Synthesis of 3-(3-carboxypropyl)-1-[2-(diethoxyphosphoryl)ethyl]-2,3-dimethyl-3H-indolium chloride

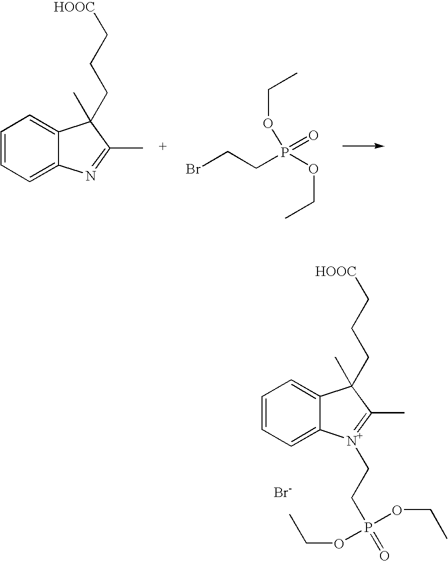

2.31 g (10.0 mmol) of 3-(3-carboxypropyl)-2,3-dimethyl-3H-indole and 2.94 g (12.0 mmol) of diethyl bromoethylphosphonate are dissolved in 10 ml of ethanol and stirred at 70° C. for 64 h. After returning to ambient temperature, the remaining liquid is extracted with two times 20 ml of water. The two combined aqueous phases are extracted twice with 20 ml of diethyl ether. The desired product, which has remained in the aqueous phase, is isolated by column chromatography (silica RP-18) with a methanol/water gradient ranging from 1:3 to 3:2 (each solvent comprising 2% by volume of 3M hydrochloric acid). The solution containing the desired product is neutralized with sodium bicarbonate and the mixture is concentrated and then deposited on a short column of silica RP-18. After having removed the inorganic salts by washing with water, the product, in the form of the sodium salt, is eluted with methanol. MS (ESI⁺): 396.2. Yield: 575 mg (12%).

### Example 3

### Synthesis of 1-[2-(diethoxyphosphoryl)-ethyl]-3,3-dimethyl-2-((1E,3E)-4-(phenylamino)buta-1,3-dienyl)-3H-indolium chloride

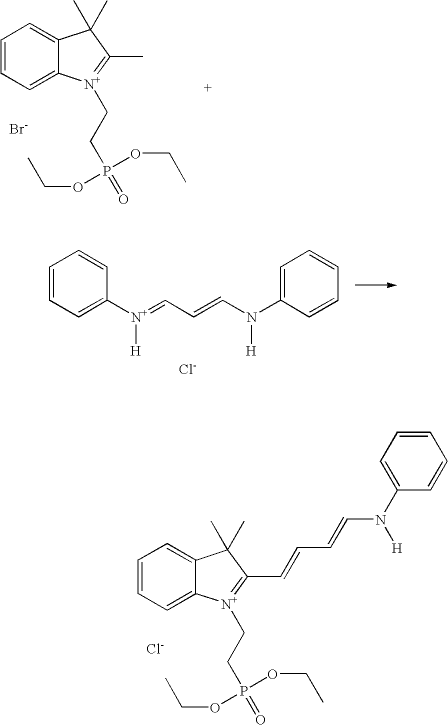

404 mg (1.0 mmol) of 1-[2-(diethoxyphosphoryl)ethyl]-2,3,3-trimethyl-3H-indolium bromide and 285 mg (1.1 mmol) of malonic aldehyde dianilide hydrochloride are dissolved in 10 ml of a mixture of acetic acid and acetic anhydride (v/v=3:2) and the mixture is stirred at 60° C. for 8 h. After returning to ambient temperature, 150 ml of water are added. The solution is deposited on a column of silica RP-18. After washing with 200 ml of water, the desired product is eluted with an acetonitrile/water gradient ranging from 1:2 to 3:2 (each solvent containing 2% by volume of 3M HCl). The solution containing the desired product is neutralized with sodium bicarbonate and the mixture is concentrated and then deposited on a short column of silica RP-18. After having removed the inorganic salts by washing with water, the product, in the form of the sodium salt, is eluted with methanol. MS (ESI⁺): 453.2. Yield: 76 mg (14%).

### Example 4

### Synthesis of 2-{(1E,3E)-5-[3-(3-carboxypropyl)-1-[2-(diethoxyphosphoryl)ethyl]-3-methyl-1,3-dihydroindol-(2E)-ylidene]penta-1,3-dienyl}-1-[2-(diethoxyphosphoryl)ethyl]-3,3-dimethyl-3H-indolium chloride (cyanine 1 in the form of the diethylphosphonoester)

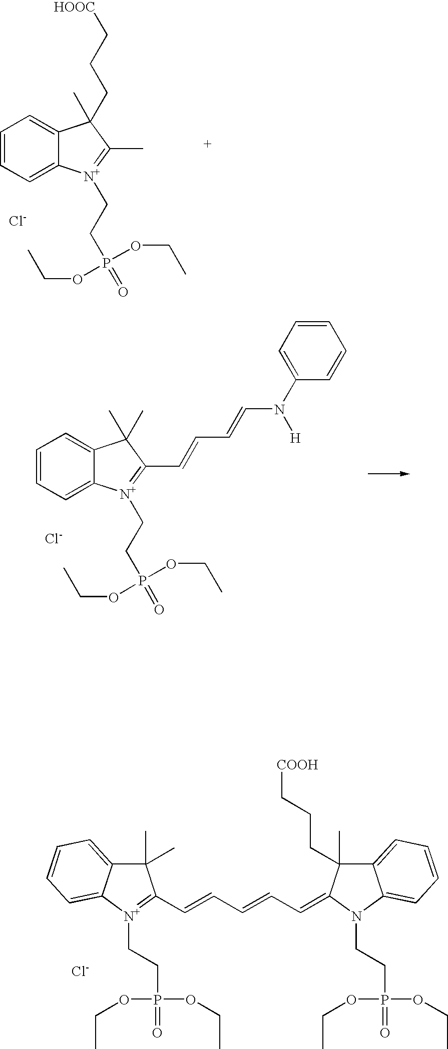

48 mg (100 μmol) of 3-(3-carboxypropyl)-1-[2-(diethoxyphosphoryl)ethyl]-2,3-dimethyl-3H-indolium chloride obtained in example 2, 53 mg (100 μmol) of 1-[2-(diethoxyphosphoryl)ethyl]-3,3-dimethyl-2-((1E,3E)-4-phenylamino)buta-1,3-dienyl)-3H-indolium chloride obtained in example 3 and 25 mg (300 μmol) of sodium acetate are dissolved in 5 ml of a mixture of acetic acid and acetic anhydride (v/v=3:2) and the mixture is stirred at 60° C. for 3 h. After concentrating under vacuum, the residue is first dissolved in 1 ml of methanol, and 2 ml of water are added. The desired compound is isolated by chromatography on a column of silica RP-18, elution being carried out with a methanol/water gradient ranging from 1:1 to 9:1. MS (ESI⁺): 755.3. Yield: 32 mg (38%).

### Example 5

### Synthesis of the Aminoethylamide Derivative Of Cyanine 1 in the Form of the Bis(Diethylphosphono Ester)

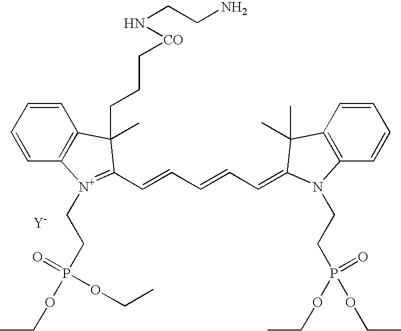

In order to be able to graft a molecule of interest, such as a substrate for an enzyme which makes possible intracellular labeling, a primary amine group is introduced.

A solution of cyanine 1 in the form of the bis(diethylphosphono ester) obtained in example 4 (160 nmol) in 100 μl of DMF is treated with 5 equivalents of diisopropylethylamine, 2.5 equivalents of tert-butyl N-(2-aminoethyl)carbamate (N-boc-ethylenediamine) and 2 equivalents of TBTU. After 24 h at ambient temperature, the starting material (t_{R}=15.7 min) is converted into a new product (t_{R}=16.8 min).

HPLC conditions: column Lichrospher® 100, RP18, 5 μm, 125 mm×4 mm; gradient of acetonitrile (ACN) in 0.1% trifluoroacetic acid (TFA) in water at 1 ml/min. Isocratic 5% ACN 3 min, linear gradient from 5% to 100% in 12 min.

The intermediate product obtained, dissolved in acetonitrile, is treated with trifluoroacetic acid (ACN/TFA: 2/1 (v/v)). The product having the deprotected amine (t_{R}=11.5 min) is isolated by semipreparative HPLC (column Grace-Vydac Protein & Peptide C18, 218TP510), flow rate 4 ml/min, with an ACN/0.1% TFA in water gradient identical to the analytical gradient.

### Example 7

### Synthesis of the N-Hydroxysuccinimide Derivative of Cyanine 1 in the Form of the Bis(Diethylphosphono Ester)

The cyanine in the form of the bis(diethylphosphono ester) obtained in example 4 (1.5 mg, 1.79 μmol) is dissolved in 70 μl of anhydrous DMF, and 1.25 μl of DIPEA and 0.55 mg of TSTU are added. By HPLC analysis (column Lichrospher® 100, RP18, 5 μm, 125 mm×4 mm; gradient of acetonitrile (ACN) in 0.1% trifluoroacetic acid (TFA) in water at 1 ml/min; isocratic 0% ACN 5 min, linear gradient from 0% to 85% in 13 min and then from 85% to 100% in 2 min), the formation is observed of a peak exhibiting a longer retention time (t_{R}=17.8 min) than the starting material (t_{R}=17.3 min). This product is isolated by semipreparative HPLC (column Grace-Vydac Protein & Peptide C18, 218TP510), flow rate 5 ml/min, with an ACN/0.1% TFA in water gradient identical to the analytical gradient. Yield: 1.1 μmol (60%).

### Example 8

### Synthesis of a Benzylguanine Derivative of Cyanine 1 in the Form of the Bis(Diethylphosphono Ester)

O⁶-[4-((13-Amino-2,5,8,11-tetraoxamidecyl)oxymethyl)-benzyl]guanine (0.46 μmol), prepared according to the protocol of the literature [Gendreizig S. et al. (2003), “Induced protein dimerization in vivo through covalent labeling”, J. Am. Chem. Soc., 125(49), 14970], in 70 μl of anhydrous DMF is treated with 0.3 μl of DIPEA and 0.46 μmol of the N-hydroxysuccinimide derivative of cyanine bis(diethylphosphono ester) prepared according to example 7. By HPLC analysis (column Lichrospher® 100, RP18, 5 μm, 125 mm×4 mm; gradient of acetonitrile (ACN) in 0.1% trifluoroacetic acid (TFA) in water at 1 ml/min. Isocratic 0% ACN 5 min, linear gradient from 0% to 70% in 13 min and then from 85% to 100% in 2 min, detection at 280 nm), the disappearance is observed of the peak corresponding to the starting guanine derivative (t_{R}=12.1 min) and the formation is observed of a peak exhibiting a longer retention time (t_{R}=16.4 min). This product is isolated by semipreparative HPLC (column Grace-Vydac Protein & Peptide C18, 218TP510), flow rate 5 ml/min, with an ACN/0.1% TFA in water linear gradient identical to the analytical gradient. Yield 0.30 μmol (66%). UV (H₂O) 643 (180 000 M⁻¹.cm⁻¹), 280 nm (16 800 M⁻¹.cm⁻¹). MS: (MALDI-TOF)/IDAA (matrix=trans-indoleacrylic acid) m/z=1185.3 (calc. 1185.36).

### Example 9

### Synthesis of a Benzylguanine Derivative of DY647

O⁶-[4-(Aminomethyl)benzyl]guanine (0.9 mg, 3.3 μmol), prepared according to the literature (Keppler S. et al. (2003), “A general method for the covalent labeling of fusion proteins with small molecules in vivo”, Nature Biotechnology, 21(1), 86-89), in 200 μl of anhydrous DMF is treated with 2.5 μmol of the N-hydroxysuccinimide derivative of DY647 (sulfonated analog of cyanine/Dyomics) at ambient temperature for 16 h. By HPLC analysis (column X-bridge, C18, 5 μm, 250 mm×4.6 mm; gradient of acetonitrile (ACN) in 0.05% trifluoroacetic acid (TFA) in water at 1 ml/min. Isocratic 10% ACN 5 min, linear gradient from 10% to 40% in 24 min, detection at 280 nm), the disappearance is observed of the peak corresponding to the starting guanine derivative (t_{R}=5.8 min) and the formation is observed of a new peak exhibiting a shorter intermediate retention time (t_{R}=19.8 min) than the peak corresponding to the reactant DY647-NHS. This product is isolated by semipreparative HPLC (column X-bridge C18, 10 mm×250 mm), flow rate 5 ml/min, with an ACN/0.05% TFA in water linear gradient identical to the analytical gradient. Yield 1.60 μmol (64%) with respect to the DY647-NHS charged. UV (H₂O) 643 (250 000 M⁻¹.cm⁻¹), 280 nm (16 200 M⁻¹.cm⁻¹). LC-MS: (ES⁺-TOF) m/z=895.4 (calc. 895.10).

### Example 10

### Synthesis of a Benzylguanine Derivative of Cyanine CY5

O⁶-[4-(Aminomethyl)benzyl]guanine (0.8 mg, 3.3 μmol) is treated with the N-hydroxysuccinimide derivative of monoreactive cyanine CY5 (2.65 μmol) (Munjumdar R. B. et al., Bioconjug. Chem., 1993, 4(2), 105-111) and 3 μmol of DIPEA in 400 μl of anhydrous DMF at ambient temperature for 90 min. By HPLC analysis (column Lichrospher® 100, RP18, 5 μm, 125 mm×4 mm; gradient of acetonitrile (ACN) in 0.05% trifluoroacetic acid (TFA) in water at 1 ml/min. Isocratic 5% ACN 5 min, linear gradient from 5% to 55% in 14 min, detection at 600 nm), the disappearance is observed of the peak corresponding to the derivative CY5-NHS (t_{R}=15.5 min) and the formation is observed of a peak exhibiting a shorter retention time (t_{R}=14.9 min) corresponding to the conjugate, which is isolated by semipreparative HPLC (column Grace-Vydac Protein & Peptide C18, 218TP510), flow rate 5 ml/min, with an ACN/0.05% TFA in water linear gradient identical to the analytical gradient). UV (H₂O) 647 (250 000 M⁻¹.cm⁻¹), 280 nm (14 000 M⁻¹.cm⁻¹). LC-MS: (ES⁺-TOF) m/z=909.15 (calc. 909.10).

### Example 11

### Test of Cell Permeability of Benzylguanine Conjugates with DY647, CY5 and Cyanine 1 in the Form of The Bis(Diethylphosphono Ester)

The cells used result from a stable CHO line expressing SnapTag in the nucleus by virtue of the NLS sequence. The compounds are added to the culture medium (DMEM medium supplemented with 10% of inactivated SVF, 30 min at 60° C.) at a concentration of 5 μM and left in contact with the cells for one hour. After the incubation, three washing operations are carried out with culture medium. An additional hour of incubation solely in the medium is carried out in order to make possible the labeling of the SnapTag by the substrate. Staining of the ring system is subsequently carried out with DAPI before the microscopic analysis.

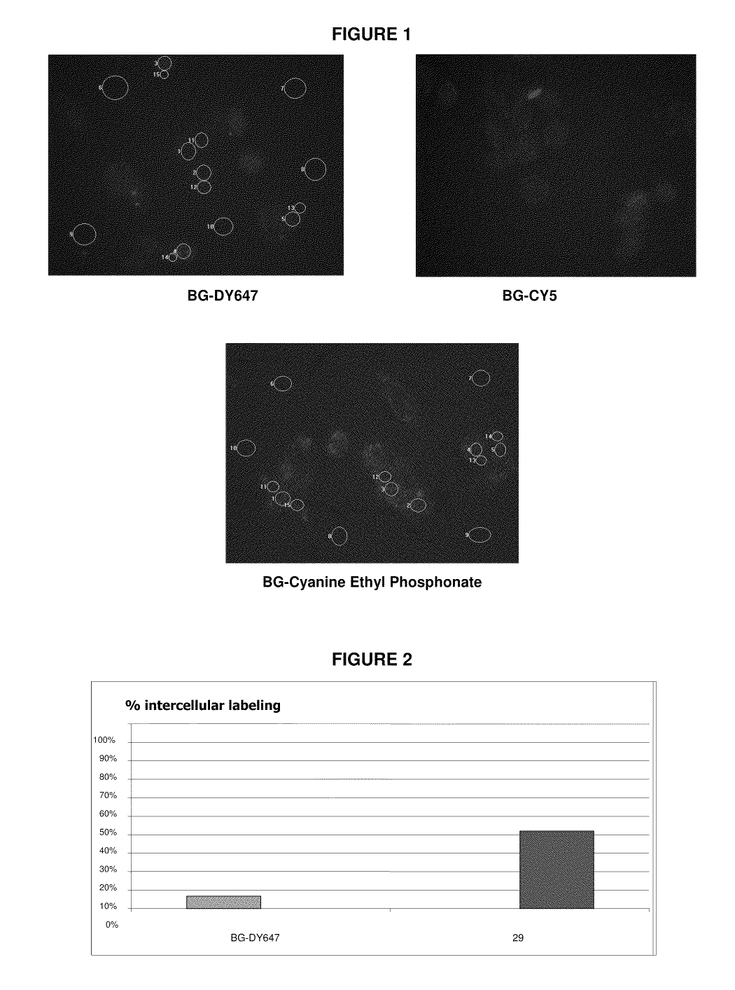

The incubation of the cells with the conjugates BG-DY647 and BG-CY5 produces images which are not very intense: on increasing the gain, fluorescence is observed solely in the form of a background noise, which shows that the conjugates tested have not passed through the plasma membrane of the cells. In contrast, the incubation of the cells with the conjugate BG-CEP (BG-Cyanine Ethyl Phosphonate), prepared according to example 8, produces an intense fluorescence located inside the cells, showing that the compound has passed through the plasma membrane. The above images are represented in FIG. 1.

### Example 12

### Synthesis of 2-{(1E,3E,5E)-5-[3-(3-carboxypropyl)-1-[2-(ethoxyphosphoryl)ethyl]-3-methyl-1,3-dihydroindol-2H-ylidene]penta-1,3-dienyl}-1-[2-(ethoxyphosphoryl)ethyl]-3,3-dimethyl-3H-indolium bromide (cyanine 1 in the form of the ethylphosphono ester)

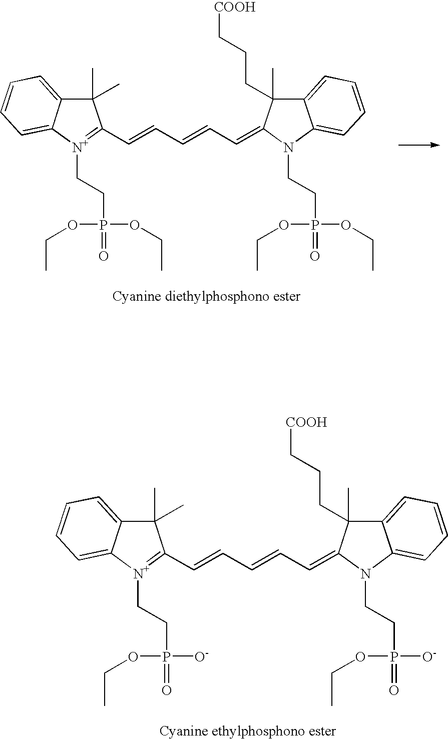

Cyanine 1 in the form of the diethylphosphono ester of example 4 (100 nmol) is dissolved in 100 μl of dichloromethane (devoid of ethanol) and then trimethylsilyl bromide (12 μmol) [McKenna C. E. et al., Tetrahedron Lett., 18, 2 (1977), 155-158] is added; after 1 h 30, a precipitate is observed, the solution is evaporated and the residue is taken up in a mixture of water and acetonitrile and purified by RP-HPLC (Shimadzu SPD-M10A diode array detector, Merck L6200A pump, Vydac 218TP510 column). A: H₂O containing 0.1% TFA B: acetonitrile. Flow rate=4 ml/min. Linear gradient from 5% ACN to 100% ACN in 15 min. The starting material (t_{R}=11.9 min) gives predominantly the desired compound carrying two ethylphosphono ester functional groups (t_{R}=0.1 min).

Peaks at t_{R}=10.6 min and at t_{R}=9.7 min, corresponding respectively to the diphosphonocyanine carrying three ethyl groups and one ethyl group, are also observed.

### Example 13

### Preparation of 2-{(1E,3E,5E)-5-[3-(5-carboxypentyl)-4-ethyl-3-methyl-5-phosphono-1,3-dihydro-2H-indol-2-ylidene]penta-1,3-dienyl}-5-phosphono-3,3-dimethyl-1-(4-ethyl)-3H-indolium derivatives

The process for the synthesis of this compound is represented in reaction scheme 1.

### 13.1. 5-Bromo-2,3,3-trimethyl-3H-indole (14a)

4-Bromophenylhydrazine hydrochloride 13 (6.5 g) and 3-methyl-2-butanone (6.4 ml) are dissolved in benzene containing a catalytic amount of acetic acid and the mixture is brought to reflux (16 h) with azeotropic entrainment of the water. The concentrated mixture is taken up in acetic acid (70 ml) and heated on an oil bath (120° C.) for 12 hours: the formation of a solid product is observed. Analysis by HPLC(RP-18, gradient of acetonitrile in water comprising 1% of TFA) shows the formation of a new product. After evaporating under vacuum, the residue is taken up in ethyl acetate and the organic phase is washed with a saturated sodium hydrogencarbonate solution, dried (MgSO₄) and evaporated. The crude product is purified by flash chromatography on silica, elution being carried out with a gradient of ethyl acetate in hexane. 5.5 g (80%) of pure product are obtained. ¹H NMR (CDCl₃) δ=7.5 (1H, d), 7.2-7.3 (2H, m), 2.3 (3H, s), 1.3 (6H, s) [Letcher R. M. et al., J. Chem. Soc. Perkin Trans., 1 (1993) 939-944].

### 13.2. 5-(Diethoxyphosphono)-2,3,3-trimethyl-3H-indole (15a)

5-Bromo-2,3,3-trimethyl-3H-indole 14a (5 g, 21 mmol) is dissolved in toluene (5 ml); degassing is carried out and then triethylamine (3.2 ml), diethyl phosphite (3 ml) and tetrakis(triphenylphosphine)palladium(0) (1.2 g) are added; the mixture is heated at 80° C. under nitrogen and with stirring for 3 h. Chromatography (flash chromatography) is carried out on silica, elution being carried out with a gradient of ethyl acetate in hexane. 3 g (48%) of pure product are obtained. ¹H NMR (CDCl₃) δ=7.7 (2H, m), 7.6 (1H, m), 4.13 (4H, m), 2.3 (3H, s), 1.3 (9H, m).

### 13.3. 5-(Diethoxyphosphono)-1-ethyl-2,3,3-trimethyl-3H-indolium iodide (16a)

5-(Diethoxyphosphono)-2,3,3-trimethyl-3H-indole 15a (3 g) is treated with ethyl iodide (10 ml) at reflux under nitrogen for 16 h. The ethyl iodide is evaporated under vacuum and the residue is triturated from hexane, collected by centrifuging and dried. It is used as is in the following reaction.

### 13.4. 5-(Phosphono)-1-ethyl-2,3,3-trimethyl-3H-indolium bromide

5-(Diethoxyphosphono)-2,3,3-trimethyl-3H-indolium iodide 16a is treated with an excess of trimethylsilyl bromide in dichloromethane (5 ml) according to the protocol of the literature [McKenna C. E. et al., Tetrahedron Lett., 18, 2 (1977), 155-158]. The completely deprotected derivative is thus obtained in the “phosphonic acid” form 17a.

### 13.5. Preparation of 3,3-dimethyl-1-ethyl-2-((1E,3E)-4-(phenylamino)buta-1,3-dienyl)-5-(phosphono)-3H-indolium chloride (18)

1-Ethyl-5-(phosphono)-2,3,3-trimethyl-3H-indolium bromide 17a (10 mmol) is treated with acetic anhydride (10 ml) and malonic aldehyde dianilide (20 mmol); reaction is allowed to take place at 60° C. for 8 h. The reaction mixture is cooled, ether (50 ml) and hexane (50 ml) are added and then the oily product (which is the desired anil) is separated by settling. Purification is carried out by chromatography (RP-18, example 3) to give the anil 18. Yield: g (40%).

### 13.6. Preparation of 5-bromo-3-(5-carboxypentyl)-2,3-dimethyl-3H-indole (14b)

4-Bromophenylhydrazine hydrochloride 13 (6.5 g) and 7-methyl-8-oxononanoic acid (6.7 g) [prepared from ethyl 2-methylacetoacetate and ethyl 6-bromohexanoate according to the process described by Leung, Wai-Yee, in WO 02/26891] are brought to reflux in acetic acid (50 ml) for 5 hours. After evaporation, the product is purified by flash chromatography on silica (yield 11 g).

### 13.7. Preparation of 3-(5-carboxypentyl)-5-(diethoxyphosphono)-2,3-dimethyl-3H-indole (15b)

5-Bromo-3-(5-carboxypentyl)-2,3-dimethyl-3H-indole (14b) is treated with diethyl phosphite as described in example 13.2 to give the derivative 15b.

### 13.8. Preparation of 3-(5-carboxypentyl)-5-(diethoxyphosphono)-2,3-dimethyl-1-ethyl-3H-indolium iodide (16b)

3-(5-Carboxypentyl)-5-(diethoxyphosphono)-2,3-dimethyl-3H-indole (15b) is treated with ethyl iodide (see example 13.3) to give the compound 16b.

### 13.9. Preparation of 3-(5-carboxypentyl)-2,3-dimethyl-1-ethyl-5-(phosphono)-3H-indolium bromide (17b)

3-(5-Carboxypentyl)-5-(diethoxyphosphono)-2,3-dimethyl-1-ethyl-3H-indolium iodide (16b) is treated with trimethylsilyl bromide (see example 13.4) to give the compound 17b.

### 13.10. Carboxylate-functionalized diphosphonocyanine (19)

### 2-{(1E,3E,5E)-5-[3-(5-Carboxypentyl)-3-methyl-4-ethyl-5-phosphono-1,3-dihydro-2H-indol-2-ylidene]penta-1,3-dienyl}-5-phosphono-3,3-dimethyl-1-(4-ethyl)-3H-indolium

The anil 18 (g, 7 mmol) and 3-(5-carboxypentyl)-2,3-dimethyl-1-(4-ethyl)-5-(phosphono)-3H-indolium bromide (17b) are dissolved in dimethylformamide (10 ml), triethylamine (1 ml) and acetic anhydride (1 ml) are added and the mixture is heated at 60° C. for 1 hour. It is cooled, ethyl ether (50 ml) is added and the precipitate is collected, taken up in dilute (2N) hydrochloric acid and then purified by chromatography (Lichroprep Merck C18), elution being carried out with 1) a sodium acetate solution (1.5 mM) and 2) distilled water; the desired product 19 is subsequently eluted with an ethanol/water (4/6) mixture. The diphosphonocyanine 19 is thus obtained in the sodium salt form. Aside from the desired diphosphonocyanine 19, the presence of a nonfunctionalized diphosphonocyanine 20 originating from the coupling of 18 with the compound 17a, which is a contaminant of 18, is also observed, these two compounds being separable by reverse phase chromatography. HPLC (Shimadzu SPD-M10A diode array detector, Merck L6200A pump, Vydac 218TP510 column). A: H₂O containing 0.1% TFA B: acetonitrile. Flow rate=4 ml/min. Linear gradient from 5% ACN to 60% ACN in 23 min. Compound 19: t_{R}=14.8 min (N.B., the compound 20 exhibits a t_{R}=14 min under the same conditions). Yield: 0.5 g (8%). MS (ES−) m/z=669.3 (100%) (ES+) m/z=671.2 (60%).

UV/Vis spectrum: λₘₐₓ (PO₄ buffer 0.1M pH 7)=649 nm (ε₆₄₉ 98 000 M⁻¹.cm⁻¹) A₆₄₉/A₆₀₄ ratio=2.6.

Fluorescence spectrum: Measurement on a Perkin-Elmer LS50 device, the samples were diluted in phosphate buffer 0.1M pH 7 containing 0.1% BSA (maximum absorbance=0.04 at the excitation wavelength). Excitation: 600 nm (10 nm). Emission (max)=670.5 nm (quantum yield=13%).

### Example 14

### Amine-Functionalized Diphosphonocyanine Tetraester (24): (See Reaction Scheme 2)

### 14.1. N-Boc derivative (21)

The carboxylated-functionalized diphosphonocyanine 19 (24 mg, 32.6 μmol) is dissolved in DMF (2 ml), and 9 μl of DIPEA and a TSTU solution (35 mg in 500 μl of DMF) are added. After 20 min, the mono-BOC derivative of ethylenediamine is added. After reacting for 1 h, purification is carried out by RP-HPLC as described in example 12. The N—BOC derivative 21 is thus obtained. MS (ES⁺) m/z=811.6 (100%), calculated for C₄₁H₅₅N₄O₉P₂.

### 14.2. N—BOC derivative of cyanine di(phosphono methyl glycolic monoester) (22)

The N—BOC derivative (21) (3 mg, 4.7 μmol) is taken up in anhydrous DMF (550 μl) and anhydrous triethylamine (distilled over calcium hydride, 14 μl, 100 μmol) and methyl bromoacetate (10 μl, 105 μmol) are added. The mixture is heated under argon at 70° C. for 16 h. The reaction mixture is purified by RP-HPLC as described in example 12 (column Vydac 218TP510, A: H₂O containing 0.1% TFA B: acetonitrile. Flow rate=4 ml/min. Linear gradient from 5% ACN to 100% ACN in 40 min). The starting material (t_{R}=16 min) gave predominantly the desired compound 22 carrying two phosphono glycolic ester functional groups (t_{R}=22.6 min). Yield 15%. MS (ES⁺) m/z=957.5 (100%), calculated for C₄₇H₆₆N₄O₁₃P₂

### 14.3. N—BOC derivative of cyanine di(phosphono methyl and methyl glycolic diester) (23)

Method A. The phosphono monoglycolic ester derivative (22) is treated with methyl iodide. The derivative 23 having each phosphonate group in the phosphono methyl and methyl glycolic diester form is thus obtained.

Method B. The phosphono monoglycolic ester derivative (22) in DMF is treated with BOP in the presence of methanol to give the product 23. The product is purified by RP-HPLC (column Vydac 218TP510, A: H₂O containing 0.1% TFA B: acetonitrile. Flow rate=4 ml/min. Linear gradient from 5% ACN to 100% ACN in 40 min). The desired compound 23 (t_{R}=31.7 min) is predominantly observed. Yield 50%. UV (H₂O/ACN, 1/1); λₘₐₓ=646.5 nm, A₆₄₆/A₆₀₀ ratio=3.7. MS (ES⁺) m/z=985.6 (100%), calculated for C₄₉H₇₁N₄O₁₃P₂.

### 14.4. Amine-functionalized cyanine di(phosphono methyl and methyl glycolic diester) (24)

The cyanine di(phosphono methyl and methyl glycolic diester) 23 is treated with trifluoroacetic acid in dichloromethane (30 min at 20° C.). After evaporating the solvent, the product is purified by RP-HPLC. The amine-functionalized cyanine tetraester 24 is thus obtained.

### Example 15

### Synthesis of a Diphosphonocyanine Tetraester Benzylguanine Derivative (26) (Reaction Scheme 2)

The cyanine 24 thus obtained is taken up in DMF containing DIPEA and treated with the NHS ester 25 [obtained by reaction of an excess of DSS (disuccinimidyl suberate) with O⁶-[4-(aminomethyl)benzyl]guanine (Keppler S. et al., (2003) Nature Biotechnology, 21(1), 86-89) in DMF; the product 25 is purified by RP-HPLC under conditions similar to those used for the purification of the cyanines in the preceding examples]. The desired compound 26 carrying two phosphono methyl and methyl glycolic ester mixed functional groups and functionalized by a benzylguanine group is obtained after purification by RP-HPLC (column Vydac 218TP510, A: H₂O containing 0.1% TFA B: acetonitrile. Flow rate=4 ml/min. Linear gradient from 5% ACN to 100% ACN in 40 min). Yield 46%. UV (H₂O/ACN, 1/1); λₘₐₓ=650 nm, A₆₄₇/A₆₀₀ ratio=3.4. MS (ES⁺) m/z=1293.8 (M-H)⁺, calculated for C₆₅H₈₇N₁₀O₄P₂

### Example 16

### Benzylguanine Derivative of Cyanine Di(Phosphono Methyl Glycolic Diester) (29): (See Reaction Scheme 3)

The phosphono monoglycolic ester derivative (22) in DMF is treated with BOP in the presence of methyl glycolate to give the product 27. The product is purified by RP-HPLC (column Vydac 218TP510, A: H₂O containing 0.1% TFA B: acetonitrile, flow rate=4 ml/min. Linear gradient from 5% ACN to 100% ACN in 40 min). The compound carrying three methyl glycolic ester functional groups (t_{R}=29.9 min) and the desired compound 27 carrying four methyl glycolic ester functional groups are observed. Yield 20%, MS (ES⁺) m/z=1102, calculated for C₅₃H₇₅N₄O₁₇P₂.

The derivative 27 is treated for 15 min in pure trifluoroacetic acid in order to remove the BOC protective group and then, after evaporation under vacuum and coevaporation with toluene, the product 28 thus obtained is taken up in DMF containing DIPEA and treated with the NHS ester 25 obtained by reaction of an excess of DSS (disuccinimidyl suberate) with O⁶-[4-(aminomethyl)benzyl]guanine in DMF (the product 25 is purified by RP-HPLC under conditions similar to those used for the purification of the cyanines in the preceding examples). The desired compound 29 carrying four methyl glycolic ester functional groups and functionalized by a benzylguanine group is obtained after purification by RP-HPLC (column Vydac 218TP510, A: H₂O containing 0.1% TFA B: acetonitrile. Flow rate=4 ml/min. Linear gradient from 5% ACN to 100% ACN in 40 min).

The product 29 is obtained after evaporation of the fractions resulting from the HPLC purification and coevaporation with toluene. It is subsequently taken up in DMSO and stored at −20° C.

Yield 70%. UV (H₂O/ACN, 1/1); λₘₐₓ=647 nm, A₆₄₇/A₆₀₀ ratio=3.5. MS (ES⁺) m/z=1409.6 (M-H)⁺, calculated for C₆₉H₉₁N₁₀O₁₈P₂.

### Example 17

### Comparison of the Quantum Yields of Different Cyanine Derivatives

The phosphate/phosphonate ester functional groups of the cyanine derivatives according to the invention are hydrolyzed in the cell by cell enzymes to give phosphate/phosphonate functional groups. The quantum yields of the cyanine derivatives carrying phosphonate groups, thus corresponding to the compounds according to the invention as present in the cell after hydrolysis of the ester functional groups, were measured in order to determine if the position of the phosphate/phosphonate ester groups influences the photophysical properties of the compounds.

| | Wavelength of | Wavelength | |
|---|---|---|---|
| | max. | of max. | Quantum |
| | absorption | emission | yield |
| | (nm) | (nm) | (%) |
| 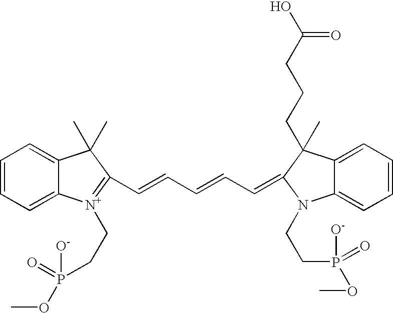 | 642 | 666 | 4% |
| 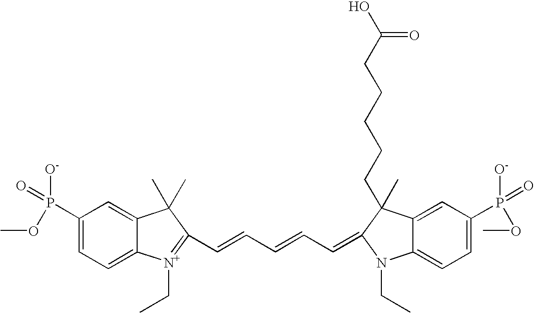 | 649 | 670 | 13% |

These results show that the cyanines comprising phosphonate functional groups on the indole rings have a quantum yield which is 3 times higher than that of those comprising phosphonates on the nitrogens of indole rings.

### Example 18

### Benzylguanine Derivative of Cyanine Di(Phosphono Methyl Glycolic Monoester) (31): (See Reaction Scheme 4)

The phosphono monoglycolic ester derivative (22) described in example 14 is treated in pure trifluoroacetic acid for 15 min in order to remove the BOC protective group and then, after evaporation under vacuum and coevaporation with toluene, the product 30 thus obtained is taken up in DMF containing DIPEA and treated with the NHS ester 25 prepared as described in example 16. The desired compound 31 carrying two methyl glycolic ester functional groups and functionalized by a benzylguanine group is obtained after purification by RP-HPLC (column Vydac 218TP510, A: H₂O containing 0.1% TFA B: acetonitrile. Flow rate=4 ml/min. Linear gradient from 5% ACN to 100% ACN in 40 min). The product is obtained after evaporation of the fractions resulting from the HPLC purification and coevaporation with toluene. It is subsequently taken up in DMSO and stored at −20° C.

Yield 53%. UV (H₂O/ACN, 1/1); λₘₐₓ=648 nm, A₆₄₈/A₆₀₀ ratio=3.2. MS (ES⁻) m/z=1263.6 (M-2H)⁻, calculated for C₆₃H₈₂N₁₀O₁₄P₂.

### Example 19

### Labeling of an Intracellular Protein by Compound 29 Added to Intact Living Cells and Detection Of the Labeling by HTRF Measurement

This example is targeted at showing that the compounds according to the invention comprising a phosphonate ester and conjugated to a substrate (benzylguanine, BG) for a “snaptag” suicide enzyme (ST26) are capable of passing through the cell membrane and of labeling a fusion protein expressed by the cell and comprising the snaptag enzyme (ST12), in contrast to the use of a cyanine not comprising phosphonate esters (DY-647).

The HTRF technique is used to test the intracellular labeling of a protein by the cyanine derivatives according to the invention, on living cells, using the recombinant protein GST-ST26-Flag produced by the cells and labeled indirectly via an anti-GST antibody conjugated with a europium trisbipyridine cryptate (anti-GST-EuTBP).

After culturing, CHO-K1 cells are transfected in the presence of lipofectamine 2000 (0.8 μl per 50 μl of transfection solution, Lipofectamine™ Transfection Reagent, Invitrogen Inc., Carlsbad, Calif.) at the rate of 50 000 cells per well in a black 96-well microplate. The transfected cells are subsequently incubated for 24 h at 37° C., 5% CO₂. The plasmid used for the transfection is a plasmid pSEM-GST-ST26-Flag (80 ng/well) obtained by introducing the sequences of GST and of Flag into the plasmid pSEMS-SNAP26m-Gateway by following the protocol of the Covalys commercial kit (Mammalian Expression Plasmid Kit pSEMSSNAP26m-Gateway® PL218), so as to induce the synthesis of a fusion protein GST-ST26-Flag by the cells. The protocol is similar to that described in the literature [Engineering Substrate Specificity of O⁶-Alkylguanine-DNA Alkyltransferase for Specific Protein Labeling in Living Cells, K. Johnsson et al., ChemBioChem, 2005, 6(7), 1263-1269].

The following day, the cells are incubated with BG-DY647 or the compound 29 (prepared according to example 16), added in the form of a solution in DMSO, in 50 μl of culture medium and so as to have a final concentration of substrate of 5 μM. The cells are incubated for 3 h at 20° C. and then washed using a culture medium.

Total labeling: the substrate is added, to a portion of the wells used to determine the “100% labeling”, at 100 nM final (BG-DY647 or the compound 29, according to the well) in a lysis buffer (cAMP kit, Cisbio) also containing an anti-GST antibody coupled to a europium cryptate (EuTBP), the anti-GST-EuTBP (1 nM final, GST tag check kit, ref. 62GSTPEB, Cisbio).

Passive labeling: only the antibody anti-GST-EuTBP (1 nM final), in the same lysis buffer, is added to the other wells used to determine the percentage of intracellular labeling (which have thus been incubated either with DY647 or with the compound 29).

Wells containing cells transfected with an “empty” plasmid, that is to say not encoding the sequence of interest, are also prepared, in order to form control wells.

The fluorescence at 665 nm is measured on an HTRF reader (RUBYstar, BMG Labtechnologies). The percentage of intracellular labeling, obtained by dividing the signal at 665 nm obtained in passive labeling by the signal at 665 nm obtained in total labeling, is represented in FIG. 2.

It is observed that the percentage of intracellular labeling in the presence of the substrate 29 is much greater than the percentage of labeling obtained in the presence of the reference substrate DY-647, which penetrates poorly into cells.

This example thus confirms that the compound 29 passes through the cell membrane and that it makes possible the labeling of an intracellular protein, which is not the case with the compound BG-DY-647 not carrying phosphate or phosphonate esters.

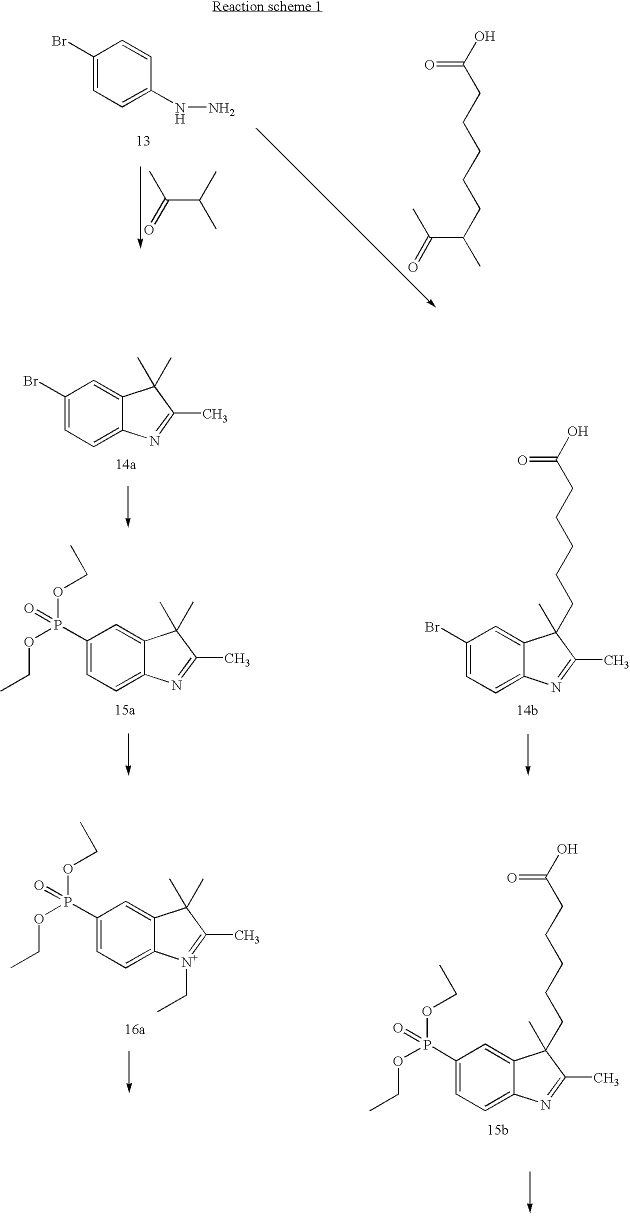

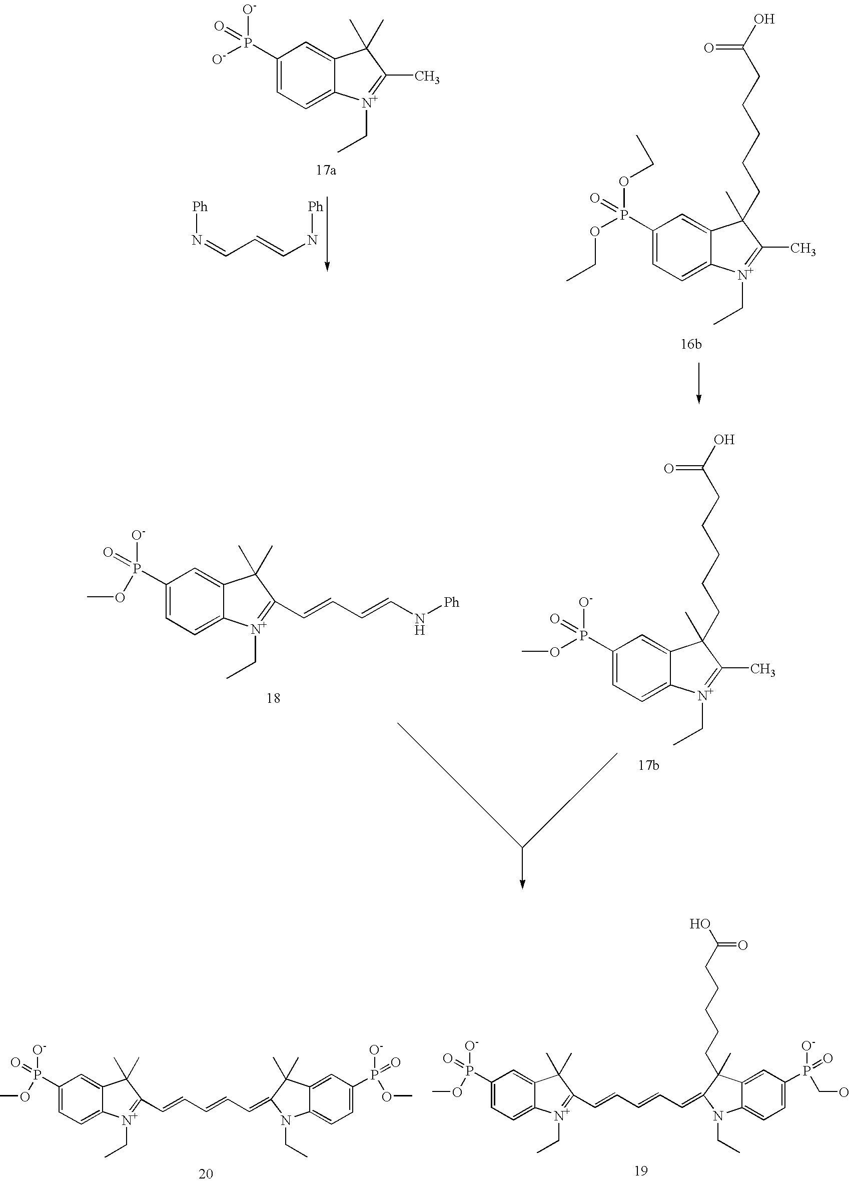

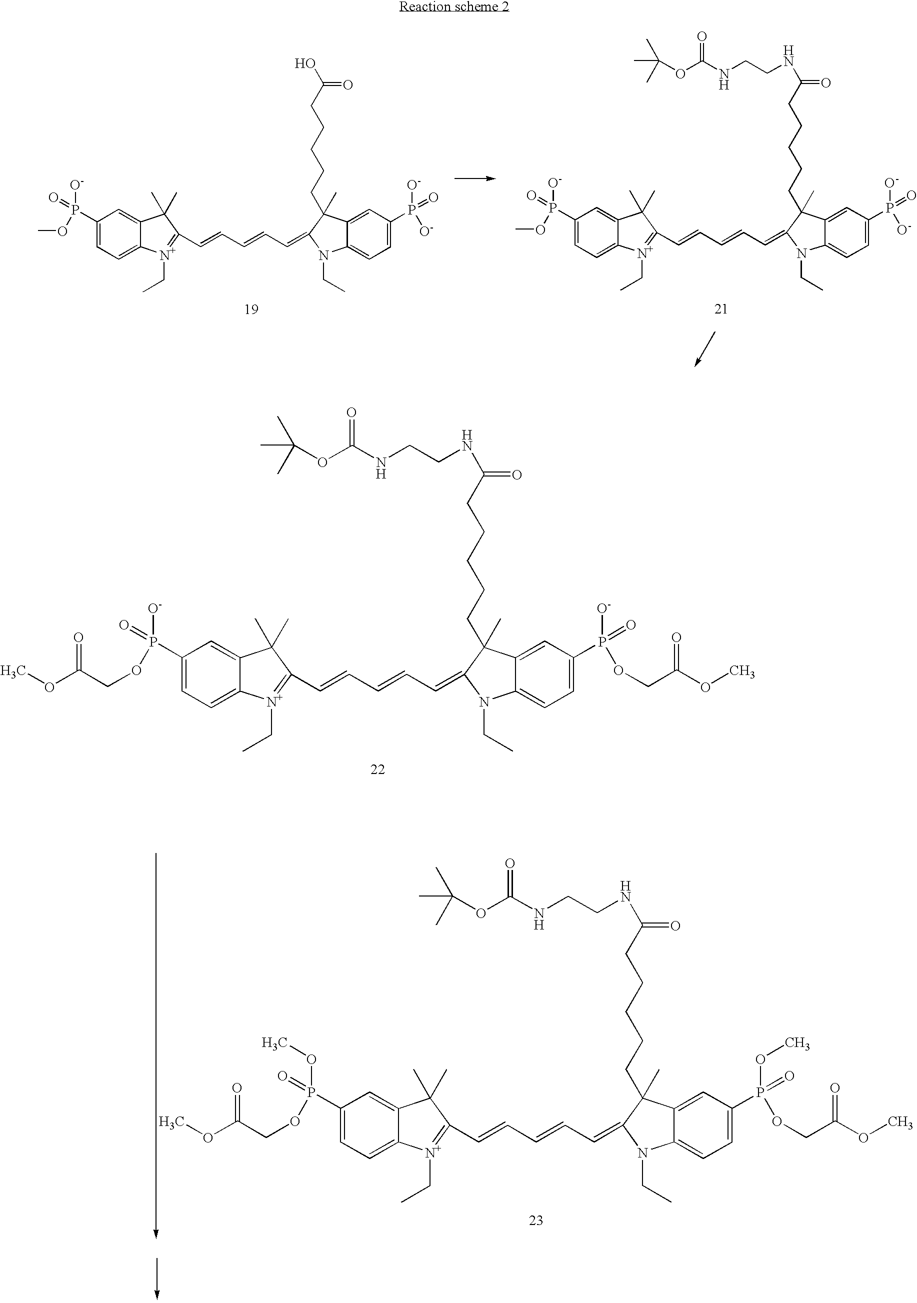

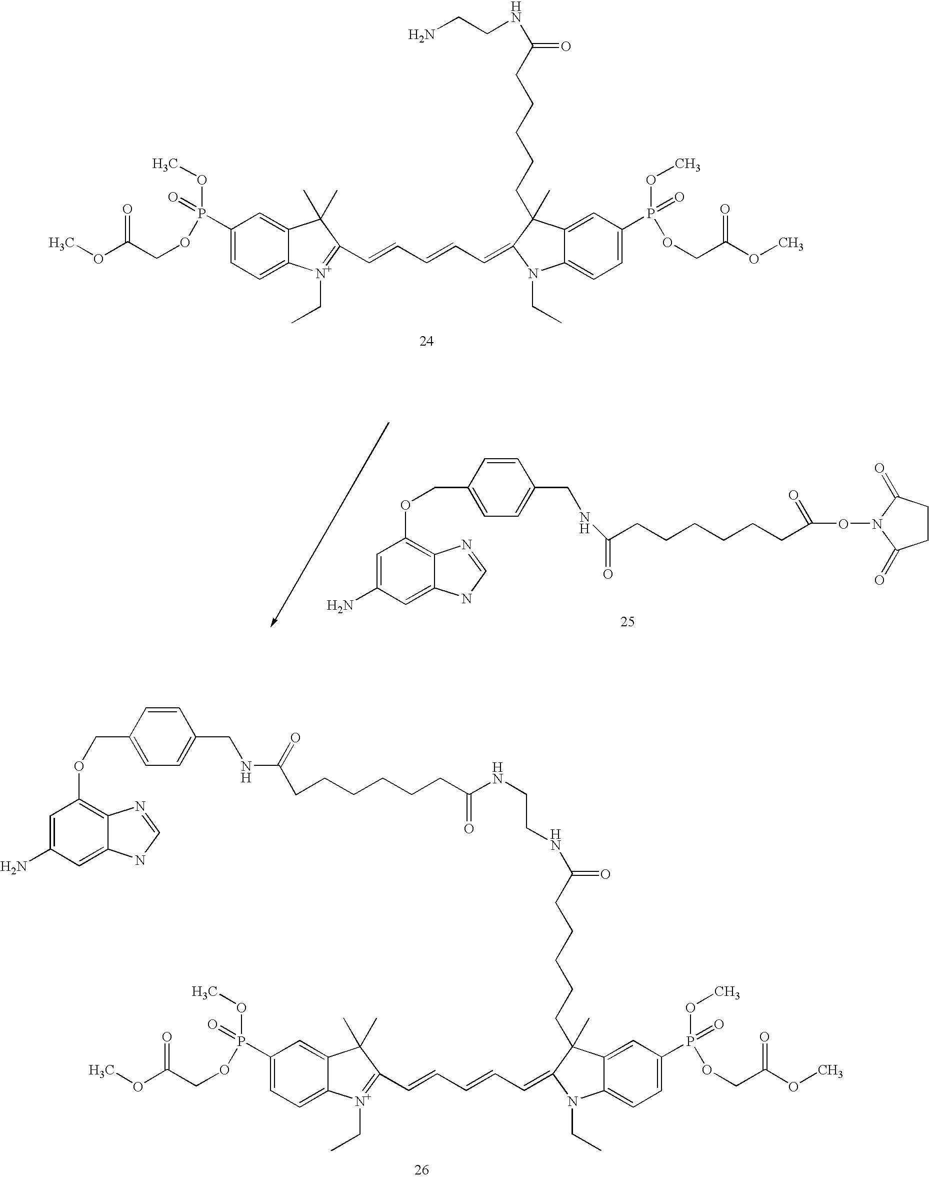

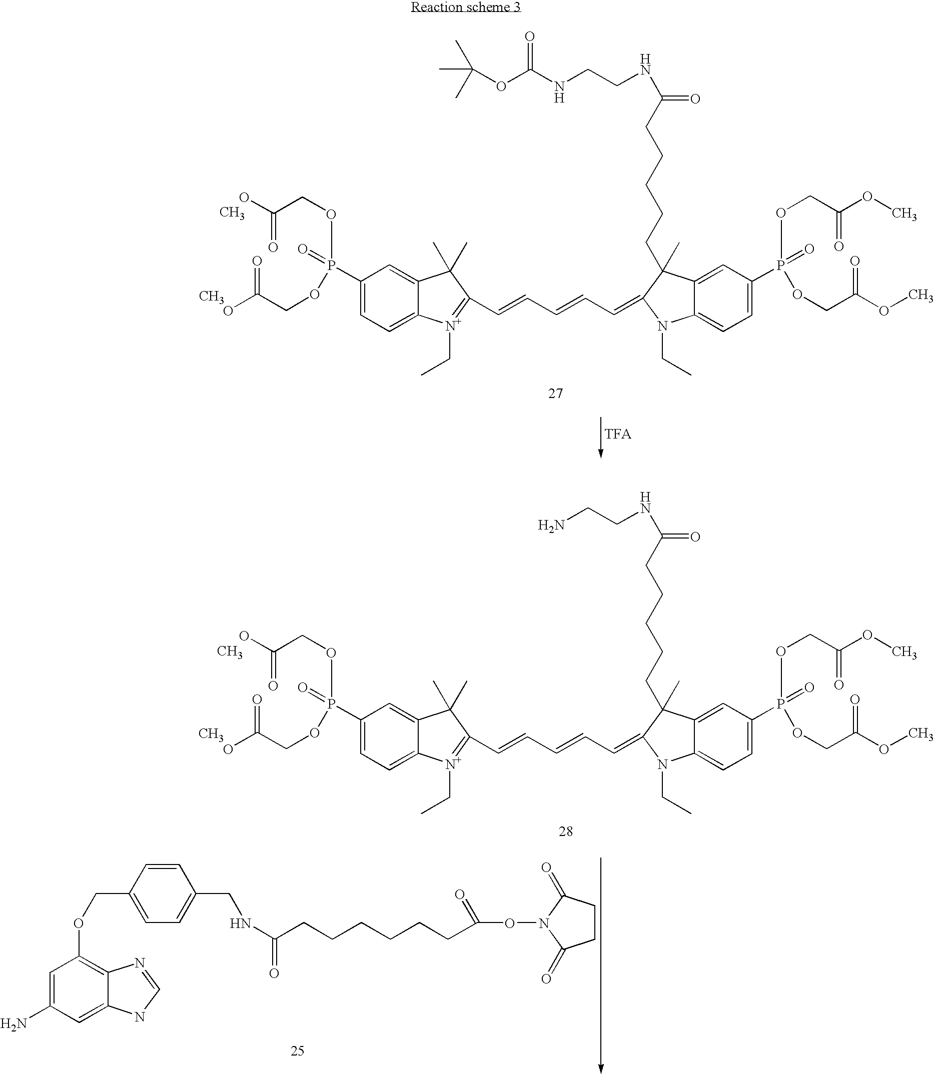

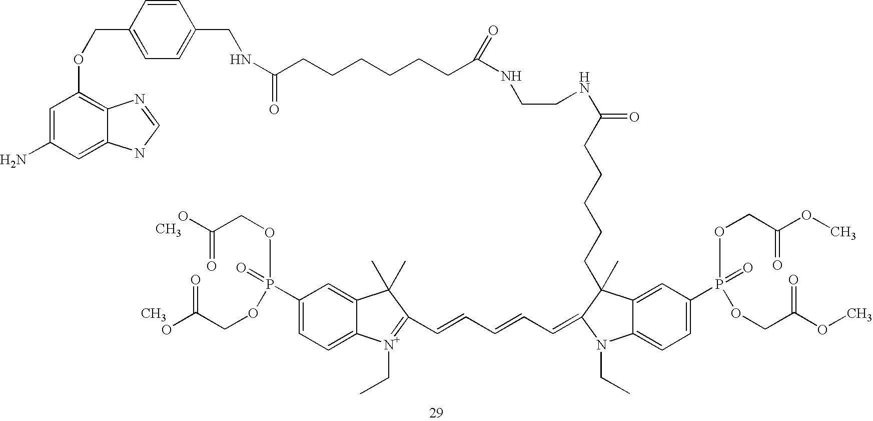

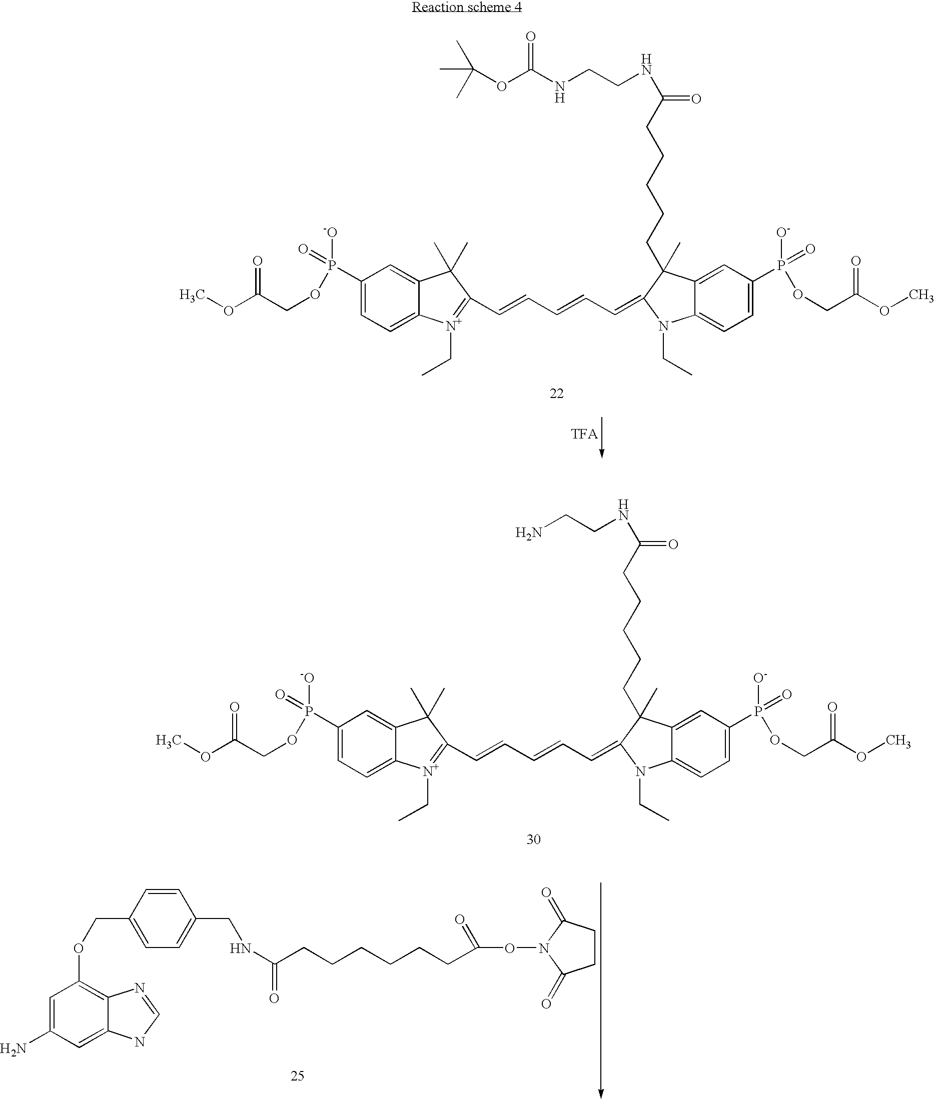

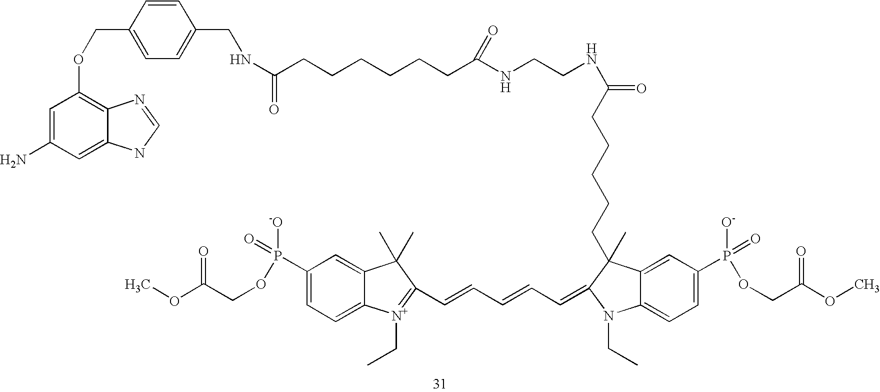

1. A cyanine derivative of formula: 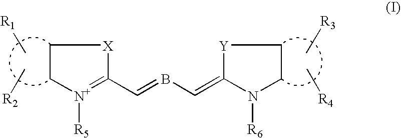
in which the dotted lines represent the atoms necessary for the formation of one or two fused aromatic rings, each ring comprising 5 or 6 carbon atoms;
R₁, R₂, R₃ and R₄ represent, independently of one another:
a hydrogen atom;
a substituted or unsubstituted C₁-C₁₅ alkyl group;
a C₁-C₆ alkoxy group;
a (C₂-C₁₂) dialkylamino group;
a C₁-C₆ alkoxycarbonyl group;
a (C₂-C₁₂) dialkylamido group;
a substituted or unsubstituted aryl, arylalkyl or aryloxy group;
a halogen atom;
a nitro group;
a group chosen from: L1-W, L2-M, L2-A or L2-G;
R₅ and R₆ represent, independently of one another:
a substituted or unsubstituted C₁-C₁₅ alkyl group;
a substituted or unsubstituted aryl or arylalkyl group;
a group chosen from: L1-W, L2-M, L2-A or L2-G;
X is chosen from: O, S or CR₇R₈;
Y is chosen from: O, S or CR₉R₁₀;
R₇, R₈, R₉ and R₁₀ independently represent:
a substituted or unsubstituted C₁-C₁₅ alkyl group;
a substituted or unsubstituted aryl, arylalkyl or aryloxy group;
a group chosen from: L1-W, L2-M, L2-A or L2-G;
R₇ and R₈ and/or R₉ and R₁₀ can also together form a ring comprising 5 or 6 atoms or a heterocycle comprising 4 to 5 carbon atoms and an oxygen atom;
B represents a polymethine bridge comprising 1 to 5 methines, in which the methine groups are individually unsubstituted or substituted by a group chosen from:
a substituted or unsubstituted C₁-C₁₅ alkyl group;
a substituted or unsubstituted aryl, arylalkyl or aryloxy group;
a nitro group;
a group chosen from: L1-W, L2-M, L2-A or L2-G;
or else two substituents of adjacent methines can together form a saturated or unsaturated hydrocarbon ring comprising 4, 5 or 6 atoms which is optionally substituted one or more times by a group chosen from:
a substituted or unsubstituted C₁-C₁₅ alkyl group;
a substituted or unsubstituted aryl, arylalkyl or aryloxy group;
a halogen atom;
a nitro group;
a group chosen from: L1-W, L2-M, L2-A or L2-G;
L1 and L2 are connecting arms;
G is a reactive group;
A is a coupling agent;
M is a conjugated molecule;
W is a phosphate or phosphonate ester chosen from: 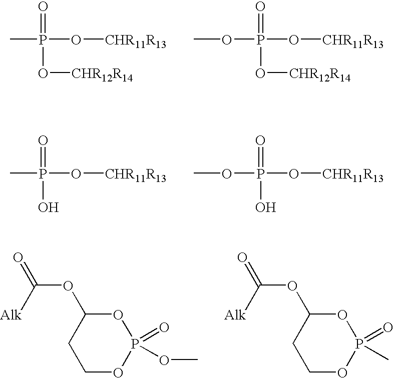
in which:
R₁₁ and R₁₂ are identical or different and are chosen from:
a hydrogen atom;
an unsubstituted C₁-C₅ alkyl group;
R₁₃ and R₁₄ are identical or different and are chosen from:
a hydrogen atom;
an unsubstituted C₁-C₁₅ alkyl group;
a C₁-C₆ alkoxycarbonyl group;
a C₁-C₆ alkylcarboxyl group;
an N,N—(C₂-C₁₂)dialkylamido group;
an amido group;
a group of formula —C—S—CO-Alk, Alk being an unsubstituted linear or branched C₁-C₄ alkyl;
R₁₁ and R₁₂ and/or R₁₃ and R₁₄ can also together form a phthalidyl group of formula: 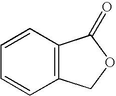
with the proviso that the cyanine derivative comprises one or two L1-W groups and one or two groups chosen from L2-A, L2-G and L2-M.

2. The derivative as claimed in claim 1, corresponding to one of the formulae below: 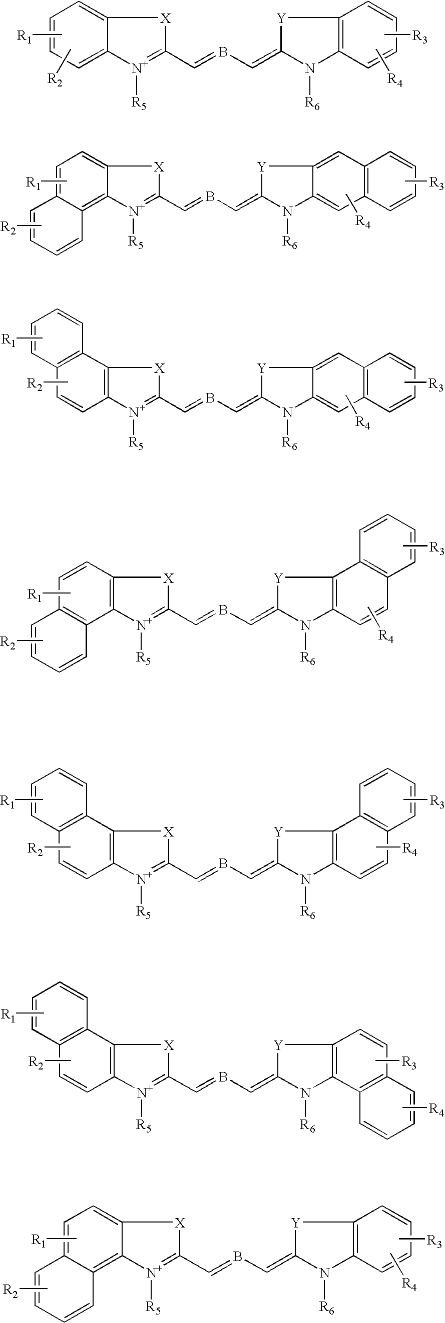 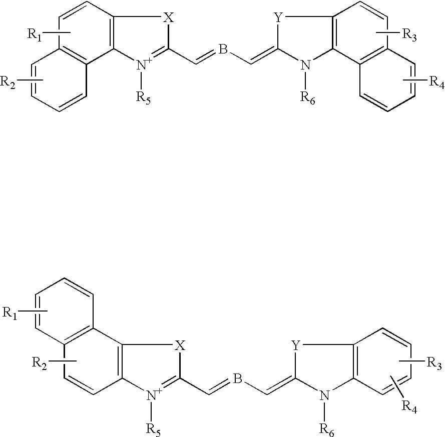
in which the R₁—R₆, X, Y and B groups are as defined in claim 1.

3. The derivative as claimed in claim 1, characterized in that the polymethine bridge B is chosen from the following formulae: 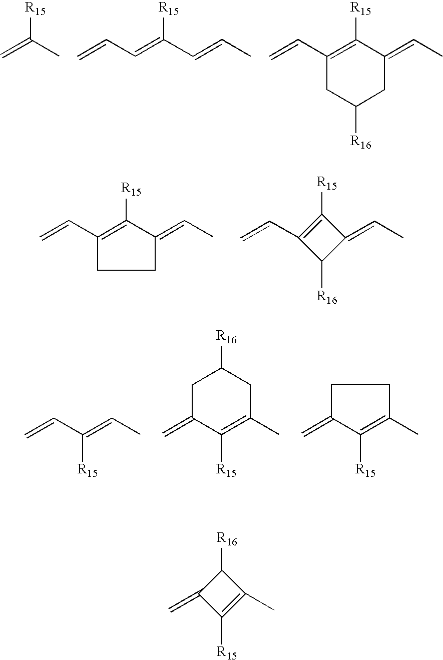
in which R₁₅ and R₁₆ are chosen from:
a hydrogen atom;
a substituted or unsubstituted C₁-C₁₅ alkyl group;
a substituted or unsubstituted aryl, arylalkyl or aryloxy group;
a halogen atom;
a nitro group;
a group chosen from: L2-M, L2-A, L2-G or L1-W.
L1 and L2 are connecting arms,
G is a reactive group,
A is a coupling agent,
M is conjugated molecule,
W is a phosphate or phosphonate ester (preferably diester).

4. The cyanine derivative as claimed in claim 1, characterized in that X is the CR₇R₈ group and/or Y is the CR₉R₁₀ group, in that one or two R₁—R₄ groups represent an L1-W group and in that one or two R₇—R₁₀ groups represent L2-A, L2-G or L2-M.

5. The cyanine derivative as claimed in claim 1, characterized in that X is the CR₇R₈ group and/or Y is the CR₉R₁₀ group, in that R₅ and/or R₆ represent an L1-W group and in that one or two R₇—R₁₀ groups represent an L2-A, L2-G or L2-M group.

6. The cyanine derivative as claimed in claim 1, characterized in that R₅ and/or R₆ represent an L1-W group and in that R₁₅ or R₁₆ represents an L2-A, L2-G or L2-M group.

7. The cyanine derivative as claimed in claim 1, characterized in that one or two R₁—R₄ groups represent an L1-W group and in that R₁₅ or R₁₆ represents an L2-A, L2-G or L2-M group.

8. The derivative as claimed in claim 1, characterized in that the W group is a phosphonate diester.

9. The cyanine derivative as claimed in claim 1, characterized in that W is a phosphonate diester formula: 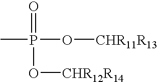
in which:
R₁₁ and R₁₂ are identical and are chosen from:
a hydrogen atom;
an unsubstituted C₁-C₅ alkyl group;
R₁₃ and R₁₄ are identical and are chosen from the following groups:
methylcarboxyl(=—O—CO—CH₃=acetoxymethyl ester);
tert-butylcarboxyl(=—O—CO—C(CH₃)₃=trimethylacetoxymethyl ester);
methyloxycarbonyl(=—CO—OCH₃=methyl glycolate ester);
methanamido(=—CO—NH₂=glycolamide ester);
N,N-dimethylmethanamido(=—CO—N(CH₃)₂=substituted glycolamide ester);
N-methylmethanamido.

10. The cyanine derivative as claimed in claim 1, characterized in that it corresponds to the formula: 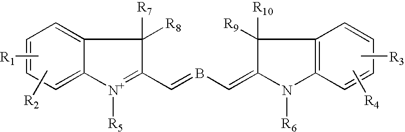
in which:
R₁ and R₃ are hydrogen atoms;
R₇, R₈ and R₉ independently represent a substituted or unsubstituted C₁-C₁₅ alkyl group;
R₅ and R₆ represent, independently of one another, a substituted or unsubstituted C₁-C₁₅ alkyl group;
B represents a polymethine bridge comprising from 1 to 5 unsubstituted methines;
R₁₀ represents a group chosen from L2-M, L2-A or L2-G;
L2 is a connecting arm;
G is a reactive group;
A is a coupling agent;
M is a conjugated molecule;
R₂ and R₄ are identical and represent an L1-W group;
L1 is a connecting arm chosen from: a single bond or a group of formula —(CH₂)ₙ—, n being an integer between 2 and 8;
W is a phosphonate diester chosen from the groups with the following formulae: 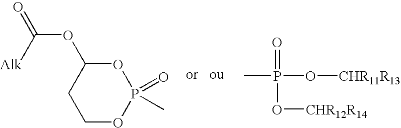
in which:
R₁₁ and R₁₂ are identical or different and are chosen from:
a hydrogen atom;
an unsubstituted C₁-C₅ alkyl group;
R₁₃ and R₁₄ are identical or different and are chosen from:
a hydrogen atom;
an unsubstituted C₁-C₁₅ alkyl group;
a C₁-C₆ alkoxycarbonyl group;
a C₁-C₆ alkylcarboxyl group;
an N,N—(C₂-C₁₂)dialkylamido group;
an amido group;
a group of formula —C—S—CO-Alk, Alk being an unsubstituted linear or branched C₁-C₄ alkyl;
R₁₁ and R₁₂ and/or R₁₃ and R₁₄ can also together form a phthalidyl group of formula:

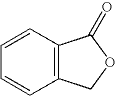

11. The cyanine derivative as claimed in claim 1, characterized in that it corresponds to the formula: 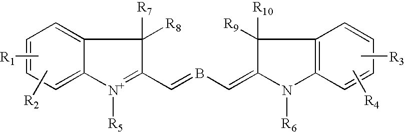
in which:
R₁, R₂, R₃ and R₄ are hydrogen atoms;
R₇, R₈ and R₉ independently represent a substituted or unsubstituted C₁-C₁₅ alkyl group;
B represents a polymethine bridge comprising from 1 to 5 unsubstituted methines;
R₁₀ represents a group chosen from L2-M, L2-A or L2-G;
L2 is a connecting arm;
G is a reactive group;
A is a coupling agent;
M is a conjugated molecule;
R₅ and R₆ are identical and represent an L1-W group;
L1 is a connecting arm chosen from a single bond or a group of formula —(CH₂)ₙ—, n being an integer between 2 and 8;
W is a phosphonate diester with a formula chosen from the following formulae: 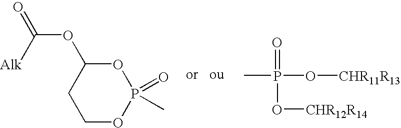
in which:
R₁₁ and R₁₂ are identical or different and are chosen from:
a hydrogen atom;
an unsubstituted C₁-C₅ alkyl group;
R₁₃ and R₁₄ are identical or different and are chosen from:
a hydrogen atom;
an unsubstituted C₁-C₁₅ alkyl group;
a C₁-C₆ alkoxycarbonyl group;
a C₁-C₆ alkylcarboxyl group;
an N,N—(C₂-C₁₂)dialkylamido group;
an amido group;
a group of formula —C—S—CO-Alk, Alk being an unsubstituted linear or branched C₁-C₄ alkyl;
R₁₁ and R₁₂ and/or R₁₃ and R₁₄ can also together form a phthalidyl group of formula:

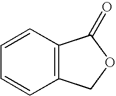

12. The cyanine derivative as claimed in claim 1, characterized in that the connecting arms L1 and L2 are chosen from a single covalent bond or a spacing arm comprising from 1 to 20 atoms other than hydrogen chosen from carbon, nitrogen, phosphorus, oxygen and sulfur atoms, this connecting group being linear or branched, cyclic or heterocyclic and saturated or unsaturated and composed of a combination of bonds chosen from: carbon-carbon bonds which can be single, double, triple or aromatic; carbon-nitrogen bonds; nitrogen-nitrogen bonds; carbon-oxygen bonds; carbon-sulfur bonds; phosphorus-oxygen bonds; phosphorus-nitrogen bonds; ether bonds; ester bonds; thioether bonds; amine bonds; amide bonds; carboxamide bonds; sulfonamide bonds; urea bonds; urethane bonds; hydrazine bonds; or carbamoyl bonds.

13. The cyanine derivative as claimed in claim 1, characterized in that the connecting group L comprises from 1 to 20 atoms other than hydrogen chosen from carbon, nitrogen, phosphorus, oxygen and sulfur atoms and additionally comprises at least one bond chosen from ether, thioether, carboxamide, sulfonamide, hydrazine, amine or ester bonds and aromatic or heteroaromatic bonds.

14. The cyanine derivative as claimed in claim 1, characterized in that the connecting group L is chosen from the following substituted or unsubstituted chains: polymethylene, arylene, alkylarylene, arylenealkyl or arylthio.

15. The cyanine derivative as claimed in claim 1, characterized in that the reactive group G is chosen from the groups derived from the following compounds: an acrylamide, an activated amine (for example, a cadaverine or an ethylenediamine), an activated ester, an aldehyde, an alkylhalide, an anhydride, an aniline, an azide, an aziridine, a carboxylic acid, a diazoalkane, a haloacetamide, a halotriazine, such as monochlorotriazine, dichlorotriazine, a hydrazine (including hydrazides), an imido ester, an isocyanate, an isothiocyanate, a maleimide, a sulfonyl halide, a thiol, a ketone, an amine, an acid halide, a hydroxysuccinimidyl ester, a hydroxysulfosuccinimidyl ester, an azidonitrophenyl, an azidophenyl, a 3-(2-pyridyldithio)propionamide or glyoxal, and in particular the groups of formula: 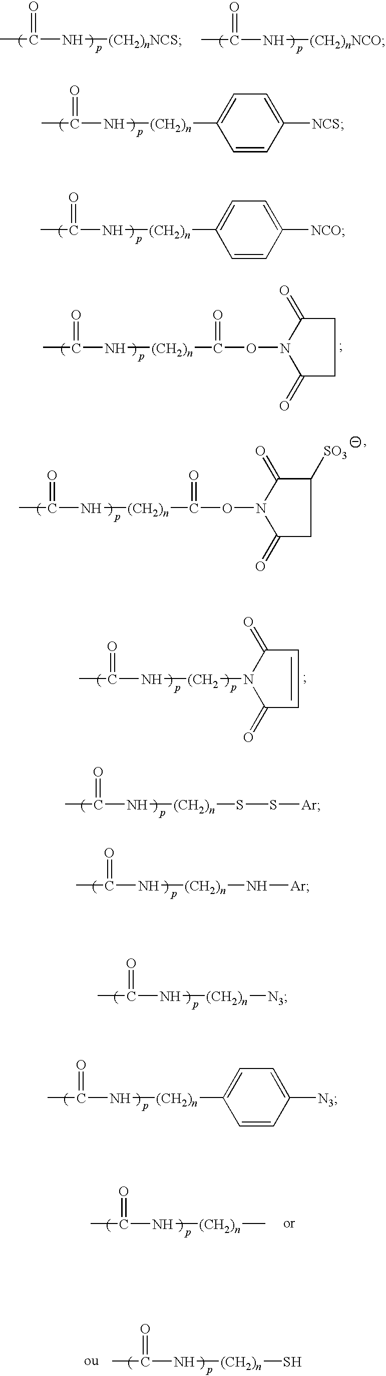
where n varies from 0 to 8 and p is equal to 0 or 1, and Ar is a 5- or 6-membered heterocycle comprising from 1 to 3 heteroatoms which is optionally substituted by a halogen atom.

16. The cyanine derivative as claimed in claim 15, characterized in that the A group is chosen from: benzylguanine or one of its derivatives which is a substrate for alkylguanine transferase, a haloalkane which is a substrate for haloalkane dehalogenase, in particular a chloroalkane, an antibody, an antibody fragment, a protein aptamer, biotin, a biarsenic compound, trimethoprim, methotrexate or SLF′.

17. The cyanine derivative as claimed in claim 1, characterized in that the M group is a biomolecule chosen from: nucleic acids, proteins, sugars, lipids, peptides, oligonucleotides, metabolic intermediates, enzymes, hormones and neurotransmitters.

18. Cyanine derivative as claimed in claim 1 comprising a fluorescent label.

19. A method for labeling a biomolecule present in a cell, characterized in that it comprises in introducing, into the extracellular medium, a compound as claimed in claim 1 comprising a coupling agent A, said biomolecule comprising a coupling domain.
